(19) United States
(12) Patent Application Publication
(10) Pub. No.: US 2025/0253052 A1
(43) Pub. Date: Aug. 7, 2025

(54) NEAR-IMPLANT PARAMETER TRANSITION

(71) Applicant: Cardiac Pacemakers, Inc., St. Paul, MN (US)

(72) Inventors: Viktoria A. Averina, Shoreview, MN (US); Pramodsingh Hirasingh Thakur, Woodbury, MN (US); Bin Mi, Arden Hills, MN (US); Mojgan Goftari, Shoreview, MN (US); Kenneth Martin Stein, Minneapolis, MN (US)

(21) Appl. No.: 19/033,060

(22) Filed: Jan. 21, 2025

Related U.S. Application Data
(60) Provisional application No. 63/550,429, filed on Feb. 6, 2024.

(51) Int. Cl. G16H 50/30; A61B 5/00; A61B 5/07; G16H 10/60

(52) U.S. Cl. CPC G16H 50/30; A61B 5/076; A61B 5/4836; A61B 5/686; A61B 5/7282; G16H 10/60

(57) ABSTRACT

Systems and methods to improve patient monitoring and device operation during a near-implant time period are disclosed, including determining a representative value of received physiologic information of a patient sensed by an implantable medical device implanted in the patient, determining a hybrid or relative baseline for the patient using the received physiologic information, and determining an indication of patient condition as a weighted function of (1) the representative value of the received physiologic information, (2) an absolute baseline, and (3) the hybrid or relative determined relative baseline, with a weight of at least one the determined absolute baseline or the determined relative baseline changing with time relative to a time of implant of the implantable medical device.

### CLAIM OF PRIORITY

This application claims the benefit of U.S. Provisional Application No. 63/550,429, filed on Feb. 6, 2024, which is hereby incorporated by reference in its entirety.

### TECHNICAL FIELD

This document relates generally to ambulatory patient monitoring, and more particularly, but not by way of limitation, to systems and methods for heart failure parameter transition in a near-implant time period.

### BACKGROUND

Ambulatory medical devices (AMDs) include implantable, subcutaneous, wearable, external, or one or more other types of medical devices having sensors configured to sense physiologic signals from a patient. Patient status or condition, including an indication of heart failure (HF), can be determined or monitored using the sensed physiologic signals. Frequent patient monitoring, such as using one or more ambulatory medical devices, can enable early detection of worsening patient status or condition or identification of patients or groups of patients having elevated risk of future adverse events.

### SUMMARY

Systems and methods to improve patient monitoring and device operation during a near-implant time period are disclosed, including determining a representative value of received physiologic information of a patient sensed by an implantable medical device implanted in the patient, determining a hybrid or relative baseline for the patient using the received physiologic information, and determining an indication of patient condition as a weighted function of (1) the representative value of the received physiologic information, (2) an absolute baseline, and (3) the hybrid or relative determined relative baseline, with a weight of at least one the determined absolute baseline or the determined relative baseline changing with time relative to a time of implant of the implantable medical device.

In other examples, systems and methods to improve patient monitoring and device operation during the near-implant time period can include determining a representative value of physiologic information of a patient sensed in a near-implant time period after implant of an implantable medical device in the patient, determining an absolute or hybrid baseline corresponding to the near-implant time period using information from at least one of an imputed baseline corresponding to a pre-implant time period preceding implant of the implantable medical device in the patient or an initial value of the received physiologic information of the patient in an initial portion of the near-implant time period, and determining an indication of patient condition as a function of the determined representative value and the determined absolute or hybrid baseline.

An example of subject matter (e.g., a medical device system) may comprise a signal receiver circuit configured to receive physiologic information of a patient sensed by an implantable medical device implanted in the patient and an assessment circuit configured to determine a representative value of the received physiologic information of the patient, to determine a hybrid or relative baseline for the patient using the received physiologic information, to determine an indication of patient condition as a weighted function of (1) the representative value of the received physiologic information, (2) an absolute baseline, and (3) the hybrid or relative determined relative baseline, with a weight of at least one the determined absolute baseline or the determined relative baseline changing with time relative to a time of implant of the implantable medical device, and to provide the determined indication of patient condition to a user or process.

In an example, which may be combined with any one or more examples described herein, the absolute baseline includes an imputed baseline stored or received by the medical device system.

In an example, which may be combined with any one or more examples described herein, to determine the representative value of the received physiologic information of the patient includes to determine a value representative of at least a portion of at least one day of the received physiologic information of the patient and to determine the relative baseline for the patient includes using information from the imputed baseline representative of a greater number of days than represented by the determined representative value.

In an example, which may be combined with any one or more examples described herein, to determine the value representative of at least a portion of at least one day of the received physiologic information includes to determine a value representative of one to three days of the received physiologic information of the patient, including a most recent one to three days, the greater number of days includes at least 30 days prior to the one to three days of the received physiologic information of the patient, prior to or including the most recent one to three days, and the assessment circuit is configured to provide an output of determined indication of patient condition to a user interface for display to the user or to a control circuit to control or adjust the process or function of the medical device system.

In an example, which may be combined with any one or more examples described herein, the assessment circuit is configured to determine the absolute baseline using information about the patient, the implantable medical device, or the received physiologic information, wherein the absolute baseline is different than the determined relative baseline.

In an example, which may be combined with any one or more examples described herein, the assessment circuit is configured to determine the absolute baseline using the received physiologic information of the patient over a time period smaller than the number of days used to determine the relative baseline.

In an example, which may be combined with any one or more examples described herein, the weight of the absolute baseline decreases in time relative to the time of implant of the medical device.

In an example, which may be combined with any one or more examples described herein, the weight of the relative baseline increases in time relative to the time of implant of the medical device.

In an example, which may be combined with any one or more examples described herein, in a first period after implant of the implantable medical device, the weight of the absolute baseline in the weighted function is greater than the weight of the determined relative baseline and, after a near-implant time period, the weight of the determined relative baseline in the weighted function is greater than the weight of the absolute baseline.

In an example, which may be combined with any one or more examples described herein, to determine the indication of patient condition as the weighted function of (1) the representative value of the received physiologic information, (2) an absolute baseline, and (3) the determined relative baseline, the assessment circuit is configured to determine, in the first period, the indication of patient condition as a function of (1) the representative value of the received physiologic information and (2) the absolute baseline, without using (3) the determined relative baseline, to determine, after the near-implant time period, the indication of patient condition as a function of (1) the representative value of the received physiologic information and (3) the determined relative baseline, without using (2) the absolute baseline, and to determine, after the first period and before the near-implant time period, the indication of patient condition as the weighted function of (1) the representative value of the received physiologic information, (2) an absolute baseline, and (3) the determined relative baseline.

An example of subject matter (e.g., a method, such as of operating a medical device system) may comprise receiving, using a signal receiver circuit, physiologic information of a patient sensed by an implantable medical device implanted in the patient, determining, using an assessment circuit, a representative value of the received physiologic information of the patient, determining, using the assessment circuit, a relative baseline for the patient using the received physiologic information, determining, using the assessment circuit, an indication of patient condition as a weighted function of (1) the representative value of the received physiologic information, (2) an absolute baseline, and (3) the determined relative baseline, with a weight of at least one the determined absolute baseline or the determined relative baseline changing with time relative to a time of implant of the implantable medical device, and providing, using the assessment circuit, the determined indication of patient condition to a user or process.

In an example, which may be combined with any one or more examples described herein, the absolute baseline includes an imputed baseline stored or received by the medical device system.

In an example, which may be combined with any one or more examples described herein, determining the representative value of the received physiologic information of the patient includes determining a value representative of at least a portion of at least one day of the received physiologic information of the patient and determining the relative baseline for the patient includes using information from the imputed baseline representative of a greater number of days than represented by the determined representative value.

In an example, which may be combined with any one or more examples described herein, determining the value representative of at least a portion of at least one day of the received physiologic information includes determining a value representative of one to three days of the received physiologic information of the patient, including a most recent one to three days, the greater number of days includes at least 30 days prior to the one to three days of the received physiologic information of the patient, prior to or including the most recent one to three days, and providing the determined indication of patient condition includes providing an output of determined indication of patient condition to a user interface for display to the user or to a control circuit to control or adjust the process or function of the medical device system.

In an example, which may be combined with any one or more examples described herein, the subject matter can include determining, using the assessment circuit, the absolute baseline using information about the patient, the implantable medical device, or the received physiologic information, wherein the absolute baseline is different than the determined relative baseline.

In an example, which may be combined with any one or more examples described herein, determining the absolute baseline includes using the received physiologic information of the patient over a time period smaller than the number of days used to determine the relative baseline.

In an example, which may be combined with any one or more examples described herein, the weight of the absolute baseline decreases in time relative to the time of implant of the medical device.

In an example, which may be combined with any one or more examples described herein, the weight of the relative baseline increases in time relative to the time of implant of the medical device.

In an example, which may be combined with any one or more examples described herein, in a first period after implant of the implantable medical device, the weight of the absolute baseline in the weighted function is greater than the weight of the determined relative baseline and, after a near-implant time period, the weight of the determined relative baseline in the weighted function is greater than the weight of the absolute baseline.

In an example, which may be combined with any one or more examples described herein, determining the indication of patient condition as the weighted function of (1) the representative value of the received physiologic information, (2) an absolute baseline, and (3) the determined relative baseline, includes determining, in the first period, the indication of patient condition as a function of (1) the representative value of the received physiologic information and (2) the absolute baseline, without using (3) the determined relative baseline, determining, after the near-implant time period, the indication of patient condition as a function of (1) the representative value of the received physiologic information and (3) the determined relative baseline, without using (2) the absolute baseline, and determining, after the first period and before the near-implant time period, the indication of patient condition as the weighted function of (1) the representative value of the received physiologic information, (2) an absolute baseline, and (3) the determined relative baseline.

An example of subject matter (e.g., a medical device system) may comprise a signal receiver circuit configured to receive physiologic information of a patient sensed in a near-implant time period after implant of an implantable medical device in the patient and an assessment circuit configured, in a near-implant mode, to determine a representative value of the received physiologic information of the patient in the near-implant time period, the representative value representative of the received physiologic information of the patient over a portion of the near-implant time period, to determine an absolute or hybrid baseline for the patient corresponding to the near-implant time period using information from at least one of an imputed baseline corresponding to a pre-implant time period preceding implant of the implantable medical device in the patient or an initial value of the received physiologic information of the patient in an initial portion of the near-implant time period, to determine an indication of patient condition as a function of the determined representative value and the determined absolute or hybrid baseline, and to provide the determined indication of patient condition in the near-implant time period to a user or process.

In an example, which may be combined with any one or more examples described herein, the assessment circuit is configured to prepend the imputed baseline corresponding to the pre-implant time period onto the received physiologic information and to determine the absolute or hybrid baseline for the patient for the near-implant time period using information from the imputed baseline corresponding to the pre-implant time period.

In an example, which may be combined with any one or more examples described herein, to determine the representative value of the received physiologic information includes to determine a value representative of at least a portion of at least one day of the received physiologic information of the patient and to determine the absolute or hybrid baseline includes to determine the initial value of the received physiologic information of the patient, the determined initial value representative of the received physiologic information of the patient in the initial portion of the near-implant time period, wherein the initial portion of the near-implant time period is a period of less than 5 days of the initial portion of the near-implant time period, including a first day after implant of the implantable medical device, and to use the determined initial value of the received physiologic information of the initial portion of the near-implant time period as the determined baseline for the near-implant time period.

In an example, which may be combined with any one or more examples described herein, the assessment circuit is configured to transition, after the near-implant time period, from the near-implant mode to a post-implant mode, the assessment circuit is configured, in the post-implant mode, to determine a relative baseline for the patient using the received physiologic information of the patient sensed after implant of the implantable medical device in the patient, without using the imputed baseline corresponding to the pre-implant time period, and the pre-implant time period, the near-implant time period, and the post-implant time period are separate, non-overlapping time periods, wherein the near-implant time period starts after implant of the implantable medical device and precedes the post-implant time period.

In an example, which may be combined with any one or more examples described herein, the assessment circuit is configured, in the near-implant time period, to determine the absolute baseline for the patient using the imputed baseline corresponding to the pre-implant time period, without using the received physiologic information of the patient sensed after implant of the implantable medical device in the patient.

In an example, which may be combined with any one or more examples described herein, the assessment circuit is configured, in the near-implant time period, to determine the hybrid baseline for the patient using the imputed baseline corresponding to the pre-implant time period and using the received physiologic information of the patient sensed after implant of the implantable medical device in the patient.

In an example, which may be combined with any one or more examples described herein, the assessment circuit is configured, in the near-implant time period, to determine the hybrid baseline for the patient as a function of the imputed baseline corresponding to the pre-implant time period and the received physiologic information of the patient sensed after implant of the implantable medical device in the patient, with a weight of the imputed baseline decreasing with time after implant and a weight of the received physiologic information of the patient increasing with time after implant.

In an example, which may be combined with any one or more examples described herein, the signal receiver circuit and the assessment circuit are components of the implantable medical device, wherein the signal receiver circuit includes a first sensor configured to sense the physiologic information of the patient.

In an example, which may be combined with any one or more examples described herein, the implantable medical device includes a first sensor configured to sense the physiologic information of the patient, wherein the signal receiver circuit is configured to receive the physiologic information from the implantable medical device.

In an example, which may be combined with any one or more examples described herein, to determine the representative value of the received physiologic information of the patient includes to determine a value representative of at least a portion of at least one day of the received physiologic information of the patient and to determine the absolute or hybrid baseline for the patient includes using information from the imputed baseline representative of a greater number of days than represented by the determined representative value.

In an example, which may be combined with any one or more examples described herein, to determine the value representative of at least a portion of at least one day of the received physiologic information includes to determine a value representative of one to three days of the received physiologic information of the patient, including a most recent one to three days, the greater number of days includes at least 30 days prior to the one to three days of the received physiologic information of the patient, prior to or including the most recent one to three days, and the assessment circuit is configured to provide an output of determined indication of patient condition to a user interface for display to the user or to a control circuit to control or adjust the process or function of the medical device system.

In an example, which may be combined with any one or more examples described herein, the assessment circuit is configured to trigger or adjust sensing by the implantable medical device or to adjust an alert threshold or a weight of an input of the function of the determined indication of patient condition after the near-implant time period using a value of the determined indication of patient condition in the near-implant time period.

In an example, which may be combined with any one or more examples described herein, the assessment circuit is configured to determine the representative value of the received physiologic information of the patient using physiologic information from a first sensor of the implantable medical device in a recovery period or the near-implant mode during the near-implant time period and using physiologic information from a second sensor of the implantable medical device after the recovery period or the near-implant time period, the second sensor different than the first sensor and the received physiologic information includes respiration information, wherein the first sensor includes an accelerometer, and wherein the second sensor includes an impedance sensor.

In an example, which may be combined with any one or more examples described herein, to receive physiologic information of the patient includes to receive separate first and second physiologic information from respective first and second sensors of the implantable medical device, to determine the absolute or hybrid baseline includes to determine first and second baselines corresponding to the respective first and second received physiologic information, to determine the representative value of the received physiologic information includes to determine first and second representative values of the received first and second physiologic information, and to determine the indication of patient condition as the function of the determined representative value and the determined absolute or hybrid baseline includes to determine the indication of patient condition as a function of the determined first value and the determined first baseline and of the determined second value and the determined second baseline, and to reduce a weight of at least one of the determined first value, the determined first baseline, the determined second value, or the determined second baseline used to determine the indication of patient condition during the near-implant time period in contrast to a corresponding weight after the near-implant time period.

An example of subject matter (e.g., a method, such as of operating a medical device system) may comprise receiving, using a signal receiver circuit, physiologic information of a patient sensed in a near-implant time period after implant of an implantable medical device in the patient, determining, using an assessment circuit, a representative value of the received physiologic information of the patient in the near-implant time period, the representative value representative of the received physiologic information of the patient over a portion of the near-implant time period, determining, using the assessment circuit, an absolute or hybrid baseline corresponding to the near-implant time period using information from at least one of an imputed baseline corresponding to a pre-implant time period preceding implant of the implantable medical device in the patient or an initial value of the received physiologic information of the patient in an initial portion of the near-implant time period, determining, using the assessment circuit, an indication of patient condition as a function of the determined representative value and the determined absolute or hybrid baseline, and providing, using the assessment circuit, the determined indication of patient condition in the near-implant time period to a user or process.

In an example, which may be combined with any one or more examples described herein, the subject matter can include prepending, using the assessment circuit, the imputed baseline corresponding to the pre-implant time period onto the received physiologic information, wherein determining the absolute or hybrid baseline includes using information from the imputed baseline corresponding to the pre-implant time period.

In an example, which may be combined with any one or more examples described herein, determining the representative value of the received physiologic information includes determining a value representative of at least a portion of at least one day of the received physiologic information of the patient and determining the absolute or hybrid baseline includes determining the initial value of the received physiologic information of the patient, the determined initial value representative of the received physiologic information of the patient in the initial portion of the near-implant time period, wherein the initial portion of the near-implant time period is a period of less than 5 days of the initial portion of the near-implant time period, including a first day after implant of the implantable medical device, and using the determined initial value of the received physiologic information of the initial portion of the near-implant time period as the determined absolute or hybrid baseline for the near-implant time period.

In an example, which may be combined with any one or more examples described herein, the subject matter can include transitioning, after the near-implant time period, the assessment circuit from a near-implant mode to a post-implant mode in a post-implant time period and determining, using the assessment circuit, a relative baseline for the patient using the received physiologic information of the patient sensed after implant of the implantable medical device in the patient, without using the imputed baseline corresponding to the pre-implant time period, wherein the pre-implant time period, the near-implant time period, and the post-implant time period are separate, non-overlapping time periods, wherein the near-implant time period starts after implant of the implantable medical device and precedes the post-implant time period.

In an example, which may be combined with any one or more examples described herein, determining the absolute baseline for the patient using the imputed baseline corresponding to the pre-implant time period includes without using the received physiologic information of the patient sensed after implant of the implantable medical device in the patient.

In an example, which may be combined with any one or more examples described herein, determining the absolute or hybrid baseline for the patient includes using the imputed baseline corresponding to the pre-implant time period and using the received physiologic information of the patient sensed after implant of the implantable medical device in the patient.

In an example, which may be combined with any one or more examples described herein, determining the absolute or hybrid baseline includes as a function of the imputed baseline corresponding to the pre-implant time period and the received physiologic information of the patient sensed after implant of the implantable medical device in the patient, with a weight of the imputed baseline decreasing with time after implant and a weight of the received physiologic information of the patient increasing with time after implant.

In an example, which may be combined with any one or more examples described herein, determining the representative value of the received physiologic information of the patient includes determining a value representative of at least a portion of at least one day of the received physiologic information of the patient and determining the absolute or hybrid baseline for the patient includes using information from the imputed baseline representative of a greater number of days than represented by the determined representative value.

In an example, which may be combined with any one or more examples described herein, determining the value representative of at least a portion of at least one day of the received physiologic information of the patient includes determining a value representative of one to three days of the received physiologic information of the patient, including a most recent one to three days, the greater number of days includes at least 30 days prior to the one to three days of the received physiologic information of the patient, prior to or including the most recent one to three days, and providing the determined indication of patient condition in the near-implant time period to the user or process to provide includes providing an output of determined indication of patient condition to a user interface for display to the user or to a control circuit to control or adjust the process or function of the medical device system.

In an example, which may be combined with any one or more examples described herein, the subject matter can include triggering or adjusting sensing by the implantable medical device or adjusting an alert threshold or a weight of an input of the function of the determined indication of patient condition after the near-implant time period using a value of the determined indication of patient condition in the near-implant time period.

In an example, which may be combined with any one or more examples described herein, receiving physiologic information of the patient includes receiving physiologic information from a first sensor of the implantable medical device and receiving physiologic information from a second sensor of the implantable medical device, determining the representative value of the received physiologic information of the patient includes using the received physiologic information from the first sensor of the implantable medical device in a recovery period or the near-implant time period and using the received physiologic information from the second sensor of the implantable medical device after the recovery period or the near-implant time period, and the received physiologic information includes respiration information, wherein the first sensor includes an accelerometer, and wherein the second sensor includes an impedance sensor.

In an example, which may be combined with any one or more examples described herein, receiving physiologic information includes receiving separate first and second physiologic information from respective first and second sensors of the implantable medical device, determining the absolute or hybrid baseline includes determining first and second baselines corresponding to the first and second received physiologic information, determining the representative value of the received physiologic information includes determining first and second representative values of the received first and second physiologic information, and determining the indication of patient condition as the function of the determined representative value and the determined absolute or hybrid baseline includes determining the indication of patient condition as the function of the determined first value and the determined first baseline and of the determined second value and the determined second baseline, and reducing a weight of at least one of the determined first value, the determined first baseline, the determined second value, or the determined second baseline used to determine the indication of patient condition during the near-implant time period in contrast to a corresponding weight after the near-implant time period.

In an example, a system or apparatus may optionally combine any portion or combination of any portion of any one or more of the examples above, may optionally combine any portion or combination of any portion of any one or more of the examples above to comprise “means for” performing any portion of any one or more of the functions or methods of the examples above, or at least one “non-transitory machine-readable medium” including instructions that, when performed by a machine, cause the machine to perform any portion of any one or more of the functions or methods of the examples above.

In an example, a system or apparatus may optionally combine any portion or combination of any portion of any one or more of the examples above, may optionally combine any portion or combination of any portion of any one or more of the examples above to comprise “means for” performing any portion of any one or more of the functions or methods of the examples above, or at least one “non-transitory machine-readable medium” including instructions that, when performed by a machine, cause the machine to perform any portion of any one or more of the functions or methods of the examples above.

This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIGS. 1-2 illustrates example relationships between sensor measurement and determined indications of patient condition in the months following a time of implant of an implantable medical device.

FIG. 3 illustrates an example method of determining an indication of patient condition in a near-implant time period.

FIG. 4 illustrates an example system.

FIG. 5 illustrates an example patient management system and portions of an environment in which the system may operate.

FIG. 6 illustrates an example implantable medical device (IMD) electrically coupled to a heart.

FIG. 7 illustrates a block diagram of an example machine upon which any one or more of the techniques (e.g., methodologies) discussed herein may perform.

### DETAILED DESCRIPTION

Ambulatory, often implantable, medical devices include, or can be configured to receive physiologic information from, one or more sensors located within, on, or proximate to a body of a patient. Physiologic information can include, among other things, one or more of: electrical information of the patient, such as cardiac electrical information (e.g., heart rate, heart rate variability, etc.), impedance information, temperature information, and in certain examples, respiration information (e.g., a respiratory rate, a respiration volume (tidal volume), etc.); mechanical information of the patient, such as cardiac acceleration information (e.g., cardiac vibration information, pressure waveform information, heart sound information, endocardial acceleration information, acceleration information, activity information, posture information, etc.), physical activity information (e.g., activity, steps, etc.), posture or position information, pressure information, plethysmograph information, and in certain examples, respiration information; chemical information; or other physiologic information of the patient.

One or more indications of patient condition can be determined using different physiologic information of the patient sensed or determined by one or more ambulatory medical devices. Determined indications of patient condition often require establishment of patient baseline information from which to measure a change in condition, in many examples requiring determination of one or more long-term measures of patient information (e.g., measures over 30 days or longer, or at least greater than 14 days, etc.), before accurate measures of such indications can be determined that are accurately reflective of actual chronic patient condition.

In addition, implant of implantable medical devices (IMDs), such as implantable cardiac defibrillator (ICD) or a cardiac resynchronization therapy defibrillator (CRT-D) devices, or insertable cardiac monitor (ICM) devices, often requires recovery or blanking periods with respect to sensed physiologic information from such devices after implant, such as to account for shifts in sensed physiologic information associated with recovery or healing from implant, as well as initial periods to establish patient baseline information (e.g., after implant or separately after periods of missing sensor data, such as if a sensor is switched off for a period of time, a device mode has changed, etc.). For example, information from an impedance sensor can be impacted by the implant procedure itself, as swelling and recovery can impact the resulting measures. Other sensor measures may be impacted as well. Certain devices are labeled to implement a blanking period (e.g., 45 days post implant), such as to not determine patient condition, report predicted events, or determine scores or provide alerts. In one example, during such a blanking period, daily values of an algorithm which requires long-term data from which to assess worsening are marked “insufficient” or “invalid”. In an example, the blanking period can be (or be considered as or equivalent to) the near-implant time period or vice versa.

Implantable cardiac device patients are at higher risk of experiencing heart failure (HF) events shortly after implant procedure (in a time period following implant) possibly due to the implant procedure itself or due to a more fragile state of the patient that precipitated the decision to implant a device. The prevalence of heart failure events near implant, coupled with the lack of reporting from devices in that same period, reduces a perceived value of the devices.

### Imputed Sensor Measurements

The present inventors have recognized, among other things, systems and methods to improve near-implant monitoring and alert of patient condition, significantly improving device performance during the near-implant time period, such as a period of time after a time of implant of an implantable medical device, until a relative baseline can be determined using information sensed by the implanted medical device over a baseline period, or alternatively until different baselines (such as described herein) merge or agree after implant. In an example, an imputed sensor value (e.g., an imputed baseline) can be prepended onto sensor measurements during the near-implant time period, such as using clinical or population values, patient-specific values, or combinations thereof, until the imputed or prepended patient baseline is fully replaced by sensed patient information. In an example, the imputed or prepended patient baseline can behave similar to an absolute threshold. In another example, absolute and relative threshold determinations can be made in parallel, in certain examples blending or combining the different determinations in a fixed or dynamic manner, creating a hybrid baseline. In another example, a modified determination can be used for the near-implant time period, modifying existing determinations to require a smaller or more minimal baseline period from which to make determinations. In certain examples, individual sensor weightings can be adjusted in a dynamic way to account for settling periods for different sensors or sensor values.

Although described herein as a near-implant time period or a blanking period, in other examples, the systems and methods described herein are similarly applicable to other initial or initialization periods, such as an initial period after a data gap (e.g., a long-term data gap), for example, after a sensor is switched off for a period and measurements are lost or missing, such that an additional initial period may be required to re-establish patient baseline information. In other examples, an initial period can follow data gaps shorter than a long-term data gap, if other physiologic information over that period has changed greater than a threshold, indicating that patient condition has changed more than a threshold amount over the data gap, or following hospitalization or treatment, etc.

Imputing values during the near-implant time period, such as using the one or more techniques described herein, can allow an algorithm which requires long-term baseline data generate valid values (e.g., change from baseline), thus providing an immediate ability to predict events.

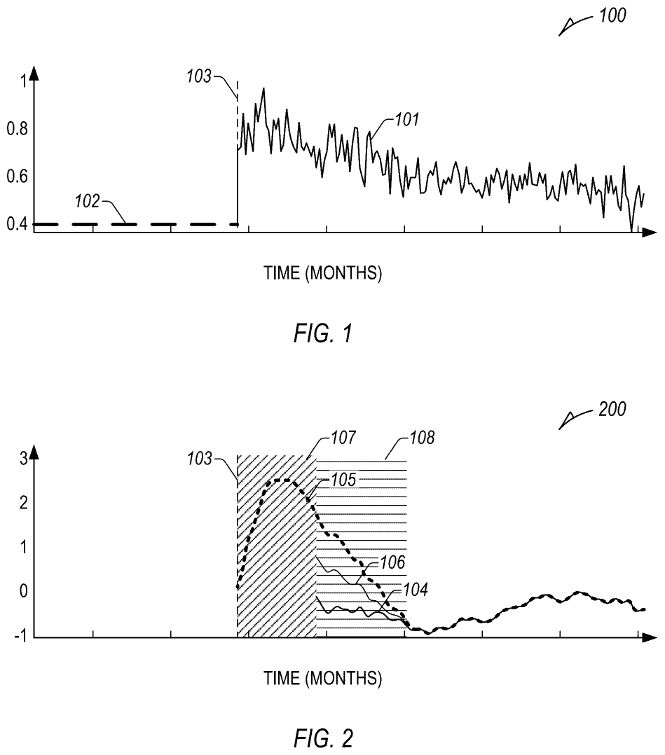

FIGS. 1-2 illustrates example relationships 100, 200 between a first sensor measurement 101 (e.g., S3, S3/S1, etc.) and a first determined indication of patient condition 104 in the months following a time of implant 103 of an implantable medical device configured to sense the first sensor measurement 101 used to determine the first indication of patient condition 104, starting roughly 30 days (1 month) after the time of implant 103.

In an example, the first indication of patient condition 104 can be determined as a function of a measure of the first sensor measurement 101, such as a representative value (e.g., a current value representative of the first sensor measurement 101 in a current period, such as a most recent daily value, a most recent short-term value, etc.) of the first sensor measurement 101 relative to (e.g., divided by) a relative baseline of the first sensor measurement 101 determined as a measure of the first sensor measurement 101 over a period of time, such as a baseline period. In an example, the representative value of the first sensor measurement 101 can include a short-term value (e.g., a measure of one or more daily values, such as a most recent daily value, or a measure of a group of most-recent daily values, such as 3, 5, or 7, generally one week or less). The relative baseline of the first sensor measurement 101 can include a long-term value of the first sensor measurement 101 (e.g., a measure of a group of most-recent daily values, such as 30 days, but generally 2 weeks or greater).

As illustrated in FIG. 2, the first indication of patient condition 104, determined using the first sensor measurement 101 alone, detected using information from a medical device after the time of implant 103, has a blanking period 107 after the time of implant 103 representative of the time required to determine the relative baseline of the first sensor measurement 101, which in the example of FIG. 1 is approximately 30 days (1 month).

FIGS. 1-2 additionally illustrates an imputed sensor measurement 102 before the time of implant 103 and a second determined indication of patient condition 105 determined using a combination of the first sensor measurement 101 and the imputed sensor measurement 102, starting roughly 30 days (1 month) before the first determined indication of patient condition 104. FIG. 2 additionally illustrates a third determined indication of patient condition 106 as a combination of the first and second determined indications of patient condition 104, 105.

In certain examples, the first, second, and third determined indications of patient condition can be determined as a function of the relative value of the first sensor measurement 101 with respect to different baselines. In an example, the first determined indication of patient condition 104 can be determined using a first baseline determined only using received physiologic information of the patient, such as the first sensor measurement 101, without the imputed sensor measurement 102 or an imputed baseline. The second determined indication of patient condition 105 can be determined using a second baseline determined using the imputed sensor measurement 102 or the imputed baseline. The third determined indication of patient condition 106 can be determined as a combination of the first and second determinations of patient condition 104, 105, or using a combination of the first and second baselines.

In an example, the imputed sensor measurement 102 can be determined based on a clinical value of the sensor measurement, such as a population average or a clinical value of other patients similar to the patient, for example, determined based on one or more measures or clinical or physiologic information of the patient, etc. In the example of FIG. 1, the imputed sensor measurement 102 has a relative value of approximately 0.4 in contrast to a representative day 1 value (after the time of implant 103) of approximately 0.7 of the first sensor measurement 101. In other examples, the imputed sensor measurement 102 can be determined as the representative day 1 value of the example sensor measurement, as a function of historical measures of the patient (e.g., an average of stored sensor measurements of the patient, an average of a percentile (e.g., a lower percentile, such as 10%, etc.) of stored sensor measurements of the patient or one or more other patients, a closest fit to stored or previously observed sensor data of the patient, etc.), or as a function of the imputed sensor measurement 102 and the clinical value.

The second determined indication of patient condition 105, in contrast to the first determined indication of patient condition 104, does not have a blanking period (a period of time without a determined value) after the time of implant 103, as an absolute or hybrid baseline for the second determined indication of patient condition 105 can be determined using the imputed sensor measurement 102 for the entire near-implant time period, using a combination of the imputed sensor measurement 102 and the first sensor measurement 101, shifting in weight or quantity until enough values of the first sensor measurement 101 are received to determine the relative baseline without the imputed sensor measurement 102, using a minimum needed days of imputed sensor measurement 102 to supplant missing information from the first sensor measurement 101 until enough values of the first sensor measurement 101 are received to determine the relative baseline without the imputed sensor measurement 102, or one or more combinations or interpolations thereof.

Although having at least some determination of patient condition during the blanking period 107 after the time of implant 103 is a distinct advantage, there are tradeoffs between the first determined indication of patient condition 104 and the second determined indication of patient condition 105. In the illustrated example, with the imputed sensor measurement 102 being lower than initial values of the first sensor measurement 101 (e.g., day 0 through day 60), the second determined indication of patient condition 105 is higher than the first determined indication of patient condition 104. Additionally, after the blanking period 107, the second determined indication of patient condition 105 remains higher than the first determined indication of patient condition 104 for a hybrid period 108 of about another 30 days (1 month) of first sensor measurements 101. As illustrated in FIG. 2, the first determined indication of patient condition 104 and the second determined indication of patient condition 105 merge at the end of the hybrid period 108, about 60 days after the time of implant 103 once a relative baseline is determined without impact of the imputed sensor measurement 102.

In one example, the near-implant time period can include the blanking period 107 and not the hybrid period 108. In another example, the near-implant time period can include both the blanking and hybrid periods 107, 108. Similarly, in one example, a post-implant time period can include the hybrid period 108, and in another example, the post-implant time period can start after the hybrid period 108, after the different indications of patient condition merge.

In an example, an imputed baseline can be determined for determinations of patient condition for the near-implant time period after the time of implant 103. For example, the imputed baseline can be determined using a population average or median of a set of clinical values, such as of other patients having received the implanted medical device, etc. In an example, the imputed baseline can be determined as an average of a percentile (e.g., a lower percentile, such as 10%, etc.) of stored sensor measurements of the patient or one or more other patients. In certain examples, the lower percentile, such as in contrast to an average, can be preferred to avoid inadvertently indicating an improved or improving patient status where one may not be warranted, reducing a likelihood of providing an inaccurate indication of patient status to a clinician or one or more other caregivers of the patient.

In other examples, observed values or patterns of the first sensor measurement 101 can be matched to historical reference data from the patient or one or more other patients. For example, a moving average (e.g., a 3-day moving average) of the first sensor measurement 101 can be determined, and the imputed baseline or the imputed sensor measurement 102 can be determined as a function of one or more stored values matching the determined moving average. In an example, one or more measurements or key metrics of the first sensor measurement 101 or one or more other measurements of the patient can be determined and matched to corresponding measurements or key metrics of a group or subgroup of patients. For example, measurements or key metrics can include one or more of: quantitative clinical measurements, such as N-terminal pro-B-type natriuretic peptide (NTproBNP), a biomarker indicative of heart failure severity; medical history data, such as prior myocardial infarction or revascularization procedures; quantitative sensor data, such as percent time the patient is in atrial fibrillation (AF burden); data clusters identified via unsupervised machine learning across multiple sensor and clinical dimensions which can be used to group similar patients; or one or more other measurements or key metrics to identify similar patients.

Alternatively, the imputed baseline or the imputed sensor measurement 102 can be determined using patient information sensed or received from one or more other devices (pre-implant devices), such as a wearable ambulatory medical device worn pre-implant configured to sense one or more of heart sounds, heart rate, respiratory rate, activity, etc., and in certain examples determine one or more baselines for such information. A difference in scale or amplitude of the different devices can be adjusted (e.g., using a correlation or correction factor) to correspond at the time of implant 103 or in a period of overlapping detection after the time of implant 103. In certain examples, the imputed sensor measurement 102 can be determined as a function of the patient information from the one or more other devices and the first sensor measurement 101. In certain examples, both pre- and post-implant devices can determine baselines or predictions after the time of implant 103 of the implantable medical device, with the implantable medical device taking over once its relative baseline is established, as implantable sensor measurements are often more accurate than wearable sensor measurements.

In an example, a multi-dimensional baseline can be determined for the imputed baseline or the imputed sensor measurement 102 using two or more aspects of patient information, such as physiologic information of the patient (e.g., sensor-based risk factors), clinical information of the patient (e.g., patient demographics, comorbidities, clinical risk factors, type of medical device, etc.), or combinations thereof. The two or more aspects can include information from two or more sensors, two or more separate measurements of one or more sensors, components of clinical information of the patient, or combinations thereof. The multi-dimensional space can include, in certain examples, two or more of: S3, S3/S1, heart rate, respiratory rate, activity, clinical information, etc. In an example, unsupervised clustering can determine subgroups having similar characteristics to the patient during the near-implant time period, and the multi-dimensional baseline can be determined using representative information for the subgroup of the patient.

In other examples, the imputed baseline or the imputed sensor measurement 102 can be determined one or more other approaches, such as determination of k-nearest neighbors, etc. In one example, a static baseline can be determined using k-nearest neighbor information for a first time period after implant (e.g., first 3 days after the time of implant) and held for the near-implant time period (e.g., for the first 30 days after implant, until the implanted medical device can establish its own baseline). In another example, a dynamic baseline can be determined for one or more time periods after implant (e.g., daily after implant, every 3 days after implant, etc.) for the near-implant time period. Representative information from different patients matched to the patient information (e.g., each day, information of or across a series of days, etc.) can be used to determine the baseline for the near-implant time period.

### Imputed Measures

Physiologic information used to determine indications of patient condition include different relative/individual or absolute/universal measures. Relative measures can include measures that vary by implant position or type of implantable medical device more than other measures, such as measures that vary between similar patients or require a certain time to stabilize (e.g., impedance measurements, etc.). In contrast, absolute measures of physiologic information can include measures that do not vary by implant position or between similar patients, and can include measures of respiration information (e.g., respiratory rate, RSBI, etc.), heart sound amplitudes or energies, activity information, heart rate information, etc. In an example, all measures used to determine indications of patient condition described herein can be imputed during the near-implant time period. In other examples, only sensors with absolute measures can be imputed, in contrast to relative measures, as relative measures can vary too greatly even among similar patients to accurately determine an imputed patient baseline for relative information that varies between patients, such as impedance, etc.

In another example, instead of imputing measures (such as imputing sensors with absolute measures described above), a requirement for a minimum number of valid measurements needed can be removed or reduced, allowing determination of indications sooner (instead of after one or more near-implant time periods, blanking periods, recovery periods, etc.). For example, whereas certain current systems and methods require an initialization period of 11 days before one or more assessment circuits can begin making risk determinations (e.g., a risk stratifier, etc.) based on one or more absolute measures (e.g., S3, respiratory rate, time active, etc.), the present inventors have recognized that it can be advantageous to remove such initialization period for absolute measures that determine risk determinations for one or more other patient conditions, balancing power requirements of measurement and detection during the recovery period with clinically relevant determination of patient information during the recovery or near-implant time period.

Although described above as being 11 days, the initialization period can be one or more other periods shorter than the near-implant time period (e.g., shorter by a factor of at least 2, 3, 5, up to or including 7, etc.), in certain examples, generally reflecting a recovery time for current physiologic information to recover to a natural level no longer reflective of patient healing from the implant procedure. In an example, the recovery period can be a set time period after implant. In other examples, recovery, or the end of the recovery period, can be detected using the received physiologic information.

The near-implant time period generally refers to the time for a relative baseline (e.g., a long-term baseline, etc.) to be established using information from the implantable medical device after implant, in certain examples additionally after the patient recovers from the implant procedure. Accordingly, the near-implant time period can be a set period after implant, or in certain examples can include a predetermined time period after the recovery period or the detected recovery.

### Blended Determinations

Determinations made with reference to established patient baselines are generally relative determinations. In contrast, imputed baselines can behave, in certain examples, more similarly to absolute thresholds, and thus, determinations made with reference to imputed baselines can be considered, in certain examples, as absolute determinations. For example, taking a current measurement in contrast to an imputed static baseline and comparing the relative change to a threshold can be similar to comparing the current measurement to an absolute threshold relative to the imputed static baseline. Hybrid baselines determined using a combination of imputed baselines and additional measurements made subsequent to the imputed baselines can initially behave similar to absolute thresholds, but the similarity lessens over time as additional measurements are received.

In one example, the imputed baseline can be used for an entire blanking period until a full patient baseline can be established, without sensor measurements impacting the imputed baseline. In another example, a hybrid patient baseline can be determined using a combination (e.g., a weighted function) of the imputed baseline and sensor measurements. In certain examples, the combination can begin after implant (e.g., a first daily value after implant, a day 1 value after an implant at day 0, etc.) as a linear combination of measured values and the imputed baseline, or the measured values can include one or more periods of adjusted weight, with a first period of sensor measurements having less weight than later sensor measurements (e.g., day 1 having a smaller weight than day 14, etc.).

In an example, the hybrid patient baseline can be determined after an initial blanking period shorter than the blanking period. For example, if the blanking period is 30 days, the initial blanking period can be substantially shorter, such as a period between 1 and 14 days (e.g., 3, 5, 7, 11 days, etc.). A longer initial blanking period can potentially extend the weight of an imputed baseline different than sensor measurements more than a shorter initial blanking period. A shorter initial blanking period can potentially introduce sensor measurements during the recovery period still impacted by the implant procedure.

In certain examples, the initial blanking period or the weight of the imputed baseline can be adjusted depending on the type of implanted medical device. For example, an implantable medical device having a less intrusive implant procedure and corresponding recovery, such as an insertable cardiac monitor, etc., can have a shorter initial blanking period or a lower weight for an imputed baseline than an implantable medical device having a comparatively more intrusive implant procedure and corresponding recovery, such as an implantable medical device having one or more leads inserted into the heart or coronary vasculature, a pacemaker, a defibrillator, or a traditional cardiac rhythm management device, etc.

In certain examples, two or more determinations can be made in parallel, such as one or more relative determinations, one or more absolute determinations, one or more hybrid determinations, etc., in a fixed manner among or between different patients, such as for one or more or all clinicians or caregivers, for example, to maintain continuity for different patients in the different determinations for the one or more or all clinicians or caregivers. In other examples, one or more of the different determinations can be dynamic, adjusted based on patient information, such as sensor measurements (e.g., sensor-based risk factors), clinical information of the patient (e.g., patient demographics, comorbidities, clinical risk factors, type of medical device, etc.), or combinations thereof.

In one example, a composite determination can be determined based on two or more of the two or more determinations, in addition to or in parallel with the separate determinations, and can be provided to a user, such as a clinician, or one or more processes. In other examples, different determinations, in certain examples including the composite determination, can be provided to the user or the clinician for review or selection.

Although discussed above as a 30-day blanking period, in other examples, depending on sensor measurements and recovery periods, blanking periods can include one or more other time periods, such as 37 days, 60 days, etc. In certain examples, if the first determined indication of patient condition 104 is a current, FDA approved determination of patient condition implanting a fixed determination based on dynamic values, the first determined indication of patient condition 104 can be implemented and used after the blanking period with one or more other determinations (e.g., the second determination of patient condition 105, etc.) used during the blanking period. In an example, the one or more other determinations can be similar to the first determined indication of patient condition 104, however, having one or more differences or changes to address the initial lack of relative baseline, such as described herein. For example, one or more time periods can be shortened to require less information (e.g., temporarily reducing relative baseline determination from 30 days to 7 days, 5 days, 3 days, etc.).

In an example, a first baseline can be determined using the first 3 days (e.g., days 1 through 3, etc.) of sensor measurements (e.g., the first sensor measurement 101, etc.). In an example, the first baseline can be used for a number of days, such as 5 days, 7 days, or the entire blanking period. In other examples, a second baseline can be determined for the subsequent 3 days (e.g., days 4 through 6, etc.), repeating every 3 days, etc. In other examples, the first baseline can be determined using a first number of days, with the second baseline determined for a second longer number of days, etc. In other examples, after the first baseline, a weighted moving average can be determined reducing a weight of previous measurements or baselines in contrast to more current sensor measurements. After the blanking period, the first determined indication of patient condition 104 can be used, in certain examples, blending the first determined indication of patient condition 104 with the one or more other determinations for a time to avoid significant movement in output associated with determination changes and not sensor measurements.

In other examples, the second determination of patient condition 105 can be used as a risk determination (e.g., a risk stratifier, etc.) for the blanking period 107, adjusting a sensitivity of one or more determinations, enabling or disabling one or more sensors, adjusting one or more alert thresholds (e.g., a higher risk can correspond to a lower alert threshold, etc.), adjusting one or more weights of one or more sensor measurements (e.g., increasing a weight of specific measures corresponding to the determined risk, etc.), adjusting one or more sensing or operation modes of one or more sensors or devices in the near-implant time period (e.g., 30 days after implant, 60 days after implant, 90 days after implant, etc.). For example, sensors most impacted by the implant procedure (e.g., impedance, activity, etc.) can be reduced in weight or excluded from determinations during the near-implant time period, or one or more other sensors less impacted (e.g., heart sounds, such as S1, S3, respiration, etc.).

In other examples, one or more sensor modes can be transitioned during the near-implant time period. For example, respiration information can be determined by an impedance sensor using changes in measured electrical impedance that correlate to movement (e.g., expansion and contraction, etc.) of the thorax during respiration, but also by acceleration information that detects movement corresponding to respiration. Whereas ambulatory monitoring of respiration is typically done using impedance information, due to sensor drift and changes during the near-implant time period, the impedance sensor can be reduced in frequency or turned off, or the sensor measurements can be excluded from respiration sensing, which can instead be determined using the activity sensor (e.g., an accelerometer) during the near-implant time period, or determined as a combination of the impedance sensor and the activity sensor during the near-implant time period, or until assessment of the respiration aspects of such sensors are determined to be in agreement. For example, one can be used, with the other checked periodically until agreement is determined (e.g., until changes in determinations from both sync), after which one determination can be omitted. In an example, respiratory rate can be detected using impedance and acceleration and agreement can be determined using a comparison of both detections. In such way, accuracy of underlying measurements can be optimized while conserving power use by the ambulatory sensors. In other examples, impedance can be replaced (e.g., turned off) in determinations of patient condition during the near-implant time period with one or more other measures that are not impacted or are less impacted by the implant procedure, such as temperature, posture, etc., and returned to use (e.g., turned on) after the near-implant time period. In certain examples, a quality metric can be determined based on such agreement or an amount of change or variance in the different measures.

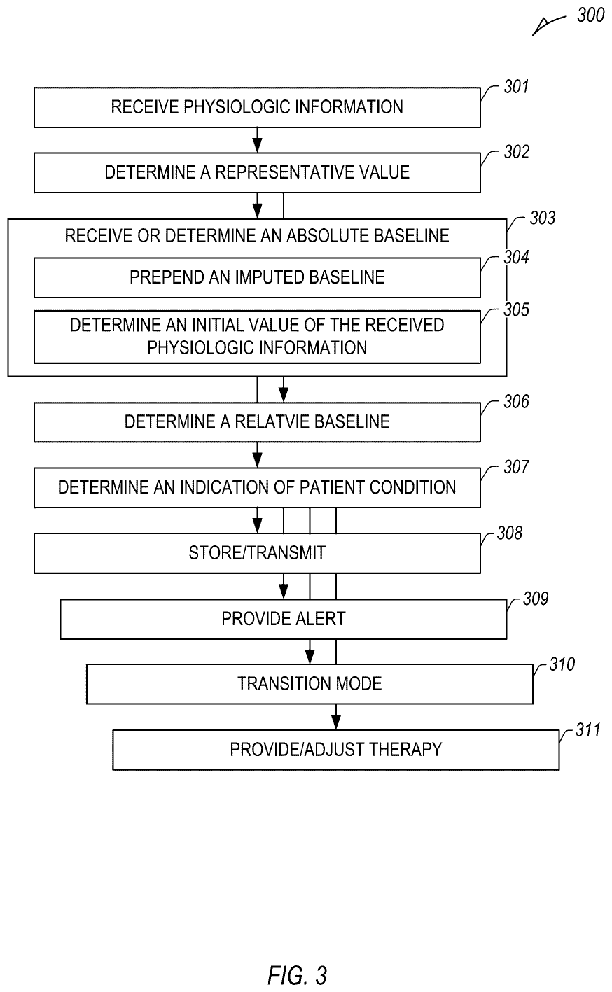

FIG. 3 illustrates an example method 300 of determining an indication of patient condition in a near-implant time period using a representative value of received physiologic information sensed from an implantable medical device in a near-implant time period after implant and an absolute or hybrid baseline. The near-implant time period can start after implant of the implantable medical device, or upon first receiving physiologic information from one or more sensors of the implantable medical device and can precede a post-implant time period.

At step 301, physiologic information of a patient sensed in a near-implant time period after implant of an implantable medical device in the patient can be received, such as using a signal receiver circuit of a sensor, an implantable medical device, an ambulatory medical device, or a component of a medical device system. The physiologic information can include one or more types of physiologic information sensed using one or sensors of an implantable medical device, such as described herein. In one example, the received physiologic information can include respiration information sensed using one or both of an accelerometer or an impedance sensor. In other examples, the received physiologic information can include other information, such as heart sound information, activity information, heart rate information, etc., sensed using one or more sensors of an implantable medical device.

At step 302, a representative value of the received physiologic information of the patient in the near-implant time period can be determined, such as by an assessment circuit, using the received physiologic information of the patient in the near-implant time period. In an example, the assessment circuit can be a component of an implantable medical device, an ambulatory medical device, or a component of a medical device system. The representative value can be representative of the received physiologic information of the patient over a portion of the near-implant time period, such as at least a portion of at least one day of the received physiologic information of the patient, a daytime value, a nighttime value, a daily value, or a value representative of more than one days, such as a short-term period (e.g., one to three days, etc.), or combinations thereof (e.g., a one- or three-day nighttime heart rate value, etc.), etc. In an example, the representative value can represent a current representative value, representative of a current or most recent time period or a time period of the physiologic information last received by the assessment circuit. For example, if the representative value is representative of one to three days of the received physiologic information of the patient, it can include a most recent one to three days of the received physiologic information, or of such most recently closed period.

In certain examples, the representative value of the received physiologic information of the patient can be determined using received physiologic information from a first sensor of the implantable medical device in a recovery period or the near-implant time period and using received physiologic information from a second sensor of the implantable medical device, different than the first sensor, after the recovery period or the near-implant time period. In other examples, the representative value of the received physiologic information of the patient can be determined using received physiologic information from a single sensor in the recovery period or the near-implant time period and using received physiologic information from multiple sensors after the recovery period or the near-implant time period, or vice versa.

Although described in FIG. 3 as one representative value, in other examples, multiple representative values of the same or different physiologic information can be determined.

At step 303, an absolute baseline (e.g., a near-implant baseline) for the patient can be received or determined corresponding to the near-implant time period, such as by the assessment circuit. In an example, the absolute baseline can be the imputed baseline for the near-implant time period. In other examples, the absolute baseline can be determined using information from the imputed baseline corresponding to a pre-implant time period preceding implant of the implantable medical device in the patient, such as in addition to or in combination with received physiologic information of the patient in an initial portion of the near-implant time period. In other examples, the absolute baseline can be determined as an initial value of the received physiologic information of the patient in the initial portion of the near-implant time period. The pre-implant time period, the near-implant time period, and the post-implant time period can be separate, non-overlapping time periods.

In an example, the absolute baseline for the patient can be determined using the imputed baseline corresponding to the pre-implant time period without using the received physiologic information of the patient sensed after implant of the implantable medical device in the patient. In other examples, the absolute baseline can include a hybrid baseline for the patient determined using the imputed baseline corresponding to the pre-implant time period and using the received physiologic information of the patient sensed after implant of the implantable medical device in the patient, changing as additional information beyond the initial portion of the near-implant time period is received. In an example, the hybrid baseline can be determined as a function of the imputed baseline and the received physiologic information, with a weight of the imputed baseline in the function decreasing with time after implant, a weight of the received physiologic information in the of the patient increasing with time after implant, or combinations thereof.

In an example, the absolute baseline for the patient can be determined using information from the imputed baseline representative of a greater number of days than represented by the determined representative value. For example, the greater number of days can include at least 30 days prior to the at least a portion of the day of the received physiologic information of the patient, prior to or including the most recent portion. If multiple different types of physiologic information is received, or multiple different representative values are determined, different baselines can be determined corresponding to each.

At step 304, an imputed baseline corresponding to the pre-implant time period can be prepended onto the received physiologic information, such as by the assessment circuit and illustrated in FIG. 1, and the absolute baseline can be determined using information from the imputed baseline corresponding to the pre-implant time period. In other examples, a value of an imputed baseline, specific to the patient or one or more clinical values, received values, or combinations thereof, can be received and used as the imputed baseline, such as for determination of the absolute baseline.

At step 305, an initial value of the received physiologic information of the patient can be determined, such as by the assessment circuit. The determined initial value can be representative of an initial portion of the received physiologic information of the patient in the near-implant time period, such as an initial period after implant. In certain examples, the initial value can be representative of a period of less than five days, such a first three days after implant, etc. In an example, the determined initial value of the received physiologic information of the initial portion of the near-implant time period can be used as the determined absolute or hybrid baseline for the near-implant time period. In other examples, the determined initial value can be used for a number of days before another value can be determined (e.g., used for 3 days, 7 days, etc.).

At step 306, a relative baseline (e.g., a post-implant baseline) can be determined for the patient, such as by the assessment circuit, and used in a post-implant time period following the near-implant time period. In certain examples, the assessment circuit can transition, after the near-implant time period (or upon determination that the absolute baseline agrees with the relative baseline) from a near-implant mode to a post-implant mode. The length of the near-implant time period can include a set time period, or in other examples detected, such as by agreement of one or more sensors (e.g., periodic impedance measurements during the near-implant time period and comparison of the periodic measure or change in periodic measures to one or more other measures, etc.), a detected stability metric (e.g., a detected reduction in drift of one or more measures after implant, etc.), a detected agreement in different determined indications of patient condition, etc. The relative baseline can be determined using the received physiologic information of the patient sensed after implant of the implantable medical device in the patient, in certain examples, without using the imputed baseline corresponding to the pre-implant time period. In certain examples, the relative baseline can be determined using information from the near-implant time period.

In certain examples, if the relative baseline is determined using information from the imputed or absolute baseline, it can be considered a hybrid baseline. In other examples, a combination baseline can be determined as a combination of a determined absolute baseline and a determined relative baseline, such as in a period before convergence of the different determined baselines.

At step 307, an indication of patient condition can be determined, such as by the assessment circuit, as a function of the determined representative value and the determined absolute or hybrid baseline, such as described herein. In certain examples, the indication of patient condition can be determined as a function of one or more determined representative values with respect to one or more determined baselines, with a weight of at least one of the determined representative values or baselines changing depending on the time period or mode of the corresponding determination. For example, a weight of a determined representative value or baseline of the near-implant time period can be lower than a weight of a determined representative value or baseline of a period after the near-implant time period. In certain examples, the determined indication of patient condition can include one or more numerical values.

In an example, the indication of patient condition can be determined as a weighted function of (1) the representative value of the received physiologic information, (2) the absolute baseline, and (3) the hybrid or relative determined relative baseline, with a weight of at least one the determined absolute baseline or the determined relative baseline changing with time relative to a time of implant of the implantable medical device. In certain examples, the weight of the absolute baseline in the weighted function in a first period after implant of the implantable medical device can be greater than the weight of the determined relative baseline, and the weight of the determined relative baseline in the weighted function after a near-implant time period can be greater than the weight of the absolute baseline.

At step 308, the determined indication of patient condition in the near-implant time period can be provided, such as by the assessment circuit, through one or more communication circuits, etc., to a user or process, such as providing an output of determined indication of patient condition to a user interface for display to the user or to a control circuit to control or adjust the process or function of the medical device system, etc. The determined indication of patient condition can be stored, such as using the assessment circuit, and transmitted, by control of the assessment circuit or using one or more communication circuits, etc., such as to one or more additional processes or components, such as an output circuit (e.g., a display, a controller for a display, etc.).

At step 309, an alert can be optionally provided, such as by the assessment circuit, for example, if the determined indication of patient condition is available for review or transmission, if one or more changes from a previous determination, such as above a threshold, etc., have been detected, if one or more detection windows of physiologic information have been determined for adjudication, if one or more comparisons to one or more previously determined indications, if a value of the determined indication of patient condition exceeds a threshold, or if a difference between the determined representative value or baseline or indication of patient condition exceeds a threshold or expected value, etc. In an example, an output can be provided of the determined indication of patient condition or a value of the determined indication of patient condition can be provided to a user interface for display to a user or to another circuit to control or adjust a process or a function of an implantable or ambulatory medical device.

At step 310, one or more modes or functions of the assessment circuit or an implantable or ambulatory medical device can be optionally adjusted based on one or more of the determined indication of patient condition or one or more corresponding values, etc. In other examples, after a set amount of time, or once one or more measures of physiologic information indicates that the patient has recovered, etc., one or more modes or functions of the implantable or ambulatory medical device can be altered to increase or decrease a power consumption or sensing or storage capability of the implantable or ambulatory medical. For example, one or more hardware limitations can be adjusted, such as to, among others: sense or receive more or less physiologic information of the patient; increase communication frequency between the implantable or ambulatory medical device and an external device (e.g., remote device, programmer, etc.), such as to increase the frequency of patient monitoring, etc.; switch to a different or more power or resource intensive monitoring algorithm; etc.

In an example, sensing by the implantable medical device can be triggered or adjusted or an alert threshold or a weight of an input of the function of the determined indication of patient condition can be adjusted after the near-implant time period using a value of the determined indication of patient condition in the near-implant time period.

At step 311, one or more therapies can be optionally provided or adjusted based on the determined indication of patient condition or one or more other measures, values, or metrics, such as described herein.

Although illustrated as a method from step 301 through step 311, in certain examples, one or more steps are options, and in other examples, different combinations or permutations of these or other steps or examples can be combined to form other methods or processes, which is also applicable to other examples discussed herein.

### Operation Modes

Ambulatory medical devices powered by rechargeable or non-rechargeable batteries, responsible for sensing physiologic signals and physiologic information of the patient, and in certain examples making determinations using such information, have to make certain tradeoffs between device battery life, or in the instance of implantable medical devices with non-rechargeable batteries, between device replacement periods often including surgical procedures, and device sensing, storage, processing, and communication characteristics, such as sensing resolution, sampling frequency, sampling periods, the number of active sensors, the amount of stored information, processing characteristics, or communication of physiologic information outside of the device.

Medical devices can include higher-power modes and lower-power modes. In certain examples, the low-power mode can include a low resource mode, characterized as requiring less power, processing time, memory, or communication time or bandwidth (e.g., transferring less data, etc.) than a corresponding high-power mode. The high-power mode can include a relatively higher resource mode, characterized as requiring more power, processing time, memory, or communication time or bandwidth than the corresponding low-power mode.

A technological problem in the art with respect to such devices exists that not all information can be stored, not all sensors can be active in a high-power or high-resolution mode, not all algorithms can be active, and not all sensed or processed information can be communicated outside of the device at all times without detrimentally impacting the lifespan of the devices. Technological solutions to such problems are often improvements in physical sensors, or alternatively in sensing and processing physiologic information in a way that improves device efficiency, extending the lifespan of the device, or to perform new determinations using existing sensors or information in a way that was not previously known, increasing the capabilities of an existing device without adding additional hardware to the device, or requiring additional sensors or hardware to be implanted in the patient. Efficiency improvements in one area can enable additional operation in another, improving the technical capabilities of existing devices having real-world constraints.

For example, physiologic information, such as indicative of a potential adverse physiologic event, can be used to transition from a low-power mode to a high-power mode. However, by the time physiologic information detected in the low-power mode indicates a possible event, valuable information has been lost, unable to be recorded in the high-power mode.

Another technological problem exists in that false or inaccurate determinations that trigger a high-power mode unnecessarily unduly limit the usable life of certain ambulatory medical devices. For numerous reasons, it is advantageous to accurately detect and determine physiologic events, and to avoid unnecessary transitions from the low-power mode to the high-power mode to improve use of medical device resources.

In an example, a change in modes can enable higher resolution sampling or an increase in the sampling frequency or number or types of sensors used to sense physiologic information leading up to and including a potential event. Different physiologic information is often sensed using non-overlapping time periods of the same sensor, in certain examples, at different sampling frequencies and power costs.

For example, ambulatory medical devices frequently contain one or more accelerometer sensors and corresponding processing circuits to determine and monitor patient acceleration information, such as, among other things, cardiac vibration information associated with blood flow or movement in the heart or patient vasculature (e.g., heart sounds, cardiac wall motion, etc.), patient physical activity or position information (e.g., patient posture, activity, etc.), respiration information (e.g., respiration rate, phase, breathing sounds, etc.), etc. In one example, heart sounds and patient activity can be detected using non-overlapping time periods of the same, single- or multi-axis accelerometer, at different sampling frequencies and power costs.

In an example, a transition to a high-power mode can include using the accelerometer to detect heart sounds throughout the high-power mode, or at a larger percentage of the high-power mode than during a corresponding low-power mode, etc. In other examples, waveforms for medical events can be recorded, stored in long-term memory, and transferred to a remote device for clinician review. In certain examples, only a notification that an event has been stored is transferred, or summary information about the event. In response, the full event can be requested for subsequent transmission and review. However, even in the situation where the event is stored and not transmitted, resources for storing and processing the event are still by the medical device.

### Physiologic Information

Heart sounds are recurring mechanical signals associated with cardiac vibrations or accelerations from blood flow through the heart or other cardiac movements with each cardiac cycle and can be separated and classified according to activity associated with such vibrations, accelerations, movements, pressure waves, or blood flow. Heart sounds include four major features: the first through the fourth heart sounds (S1 through S4, respectively). The first heart sound (S1) is the vibrational sound made by the heart during closure of the atrioventricular (AV) valves, the mitral valve and the tricuspid valve, and the opening of the aortic valve at the beginning of systole, or ventricular contraction. The second heart sound (S2) is the vibrational sound made by the heart during closure of the aortic and pulmonary valves at the beginning of diastole, or ventricular relaxation. The third and fourth heart sounds (S3, S4) are related to filling pressures of the left ventricle during diastole. An abrupt halt of early diastolic filling can cause the third heart sound (S3). Vibrations due to atrial kick can cause the fourth heart sound (S4). Valve closures and blood movement and pressure changes in the heart can cause accelerations, vibrations, or movement of the cardiac walls that can be detected using an accelerometer or a microphone, providing an output referred to herein as cardiac acceleration information.

Respiration information can include, among other things, a respiratory rate (RR) of the patient, a tidal volume (TV) of the patient, a rapid shallow breathing index (RSBI) of the patient, or other respiratory information of the patient. The respiratory rate is a measure of a breathing rate of the patient, generally measured in breaths per minute. The tidal volume is an aggregate measure of respiration changes, such as detected using measured changes in thoracic impedance, etc. The RSBI is a measure (e.g., a ratio) of respiratory frequency relative to (e.g., divided by) tidal volume of the patient. The nHR is a measure of heart rate (HR) of the patient at night, either in relation to sensing patient sleep or using a preset or selectable time of day corresponding to patient sleep. In certain examples, respiration information of the patient can be determined using changes in impedance information and accordingly can be considered electrical information, but different than cardiac electrical information. In other examples, respiration information of the patient can be determined using changes in activity or acceleration information and accordingly can be considered mechanical information.

Physiologic metrics, as described herein, or measures or indications of physiologic information, can include one or more different measures of rate, amplitude, energy, etc., of different physiologic information over one or more time periods, such as representative daily values, etc. For example, heart sound metrics can be determined for each heart sound (e.g., the first heart sound (S1) through the fourth heart sound (S4), etc.) and can include an indication of an amplitude or energy of a specific heart sound for a specific cardiac cycle, or a representation of a number of cardiac cycles of the patient over a specific time period. Daily metrics can be determined representative of an average daily value for the patient, either corresponding to a waking time or a 24-hour period, etc. Respiration metrics can include, among other things, a mean or median respiration rate, binned values of rates, and a representative value of specific rate bins, etc. Heart rate metrics can include an average nighttime heart rate, a minimum nighttime heart rate, heart rate at rest, etc.

The activity information can include an activity measurement of the patient, such as detected using an accelerometer, a posture sensor, a step counter, or one or more other activity sensors associated with an ambulatory medical device. Activity may be used to gate other physiologic measurements such as heart rate or respiration rate so that the change in these metrics with increased patient activity may be used to infer patient cardiovascular and metabolic status including measurement of oxygen consumption. The impedance information can include, among other things, thoracic impedance information of the patient, such as a measure of impedance across a thorax of the patient from one or more electrodes associated with the ambulatory medical device (e.g., one or more leads of an implantable medical device proximate a heart of the patient and a housing of the implantable medical device implanted subcutaneously at a thoracic location of the patient, one or more external leads on a body of the patient, etc.). In other examples, the impedance information can include one or more other impedance measurements associated with the thorax of the patient, or otherwise indicative of patient thoracic impedance.

The temperature information can include an internal patient temperature at an ambulatory medical device, such as implanted in the thorax of the patient, or one or more other temperature measurements made at a specific location on the patient, etc. The temperature information can be detected using a temperature sensor, such as one or more circuits or electronic components having an electrical characteristic that changes with temperature. The temperature sensor can include a sensing element located on, at, or within the ambulatory medical device configured to determine a temperature indicative of patient temperature at the location of the ambulatory medical device.

In contrast to and separate from the electrical or mechanical information discussed above, the chemical information can include information about one or more chemical properties of blood, interstitial space (e.g., the space between cells, such as including interstitial fluid), or other tissue (e.g., muscle tissue, fat tissue, organ tissue, etc.) of the patient, such as information indicative of or including one or more of a glucose level, pH level, dissolved gas level (e.g., oxygen, carbon dioxide, carbon monoxide, etc.), electrolyte level (e.g., sodium, potassium, calcium, etc.), organic compound level (e.g., lactate, cholesterol, hemoglobin, creatinine, etc.), or biologic compound level (e.g., enzymes, antibodies, receptors, etc.), etc. The chemical information may be measured by one or more of an electrical sensor, mechanical sensor, electrochemical sensor, biosensor (e.g., enzyme biosensor, etc.), ion-selective electrode sensor, optical sensor, etc. In an example, the chemical information may include potassium information (e.g., one or more of interstitial potassium information, serum potassium information, etc.), creatinine information (e.g., one or more of interstitial creatinine information, serum creatinine information, etc.), or combinations thereof.

In certain examples, interstitial chemical information, such as one or more chemical levels in an interstitial space (e.g., a space between one or more of connective tissue, muscle fibers, nervous tissue, etc.) or of interstitial fluid, etc., can be indicative of serum chemical information. For example, potassium may move between cells or tissue and interstitial fluid (e.g., a change in interstitial potassium level may be followed by or reflective of a change in serum potassium level or vice versa), such that chemical information on serum potassium can include interstitial potassium. In certain examples, one of interstitial or serum chemical information can lead or lag the other, such that a change in one can indicate a worsening patient condition is detectable before the other. In one example, interstitial potassium information can lead serum potassium information as an indicator of electrolyte imbalance.

In certain examples, an alert state (e.g., an in-alert state, an out-of-alert state, a priority alert state, etc.) of the patient can be adjusted or determined using chemical information of the patient, such as to increase a sensitivity or specificity of alert state determination, reduce false positive alert state determinations, alert state transitions or adjustments, or otherwise reduce storage or transmission of physiologic information associated or transitions associated with false positive alert state determinations, and power and processing resources associated with the same. In an example, the alert state can be determined using a comparison of a value of the health index (e.g., a numerical value, etc.) to one or more fixed or adaptable alert thresholds (e.g., based at least in part on one or more relative factors, such as measurements from the patient over the past 30 days, etc.). In an example, the alert state can be provided to a user interface for display to a user or to a control circuit to control or adjust a process or function of the system. In an example, the alert state can include one or more of an indication, recommendation, or instruction to perform one or more actions (e.g., administer or provide a drug or class of drug, adjust or optimize a guideline-directed medical therapy (GDMT), etc.). For, example, a GDMT may advise administration of a quantity of a drug or a rate of increase in a dosage, etc. In an example, determination of an in-alert or priority alert state can trigger an indication or instruction to administer or provide a specific class of diuretic or to deviate from GDMT (e.g., increase GDMT above a standard recommendation, hold GDMT at a standard recommendation, hold GDMT at a current level, decrease GDMT below a standard recommendation, increase a dosage or rate of increase of a drug, reduce a dosage or rate of decrease of a drug, etc.).

In certain examples, the techniques described above or herein can be used in various combinations or permutations. For example, combinations or permutations of techniques described above or herein can be selected based upon patient history, patient treatment (e.g., in-patient care, out-patient care, etc.), clinician input, etc.

As used herein, high and low (or high, medium, and low, etc.) can be relative or categorical terms, in certain examples with respect to clinical or population values, patient-specific values (e.g., a representative value, such as a current value, with respect to a short- or long-term range of values, etc.), or combinations thereof. For example, a high value can include a value in an upper percentage (e.g., at or above an upper quartile, etc.) of values experienced by the patient over respective time periods, such as one or more of a short-term range (e.g., having a period between 1 week and 3 months, such as 1 month, etc.), a long term range (e.g., having a period greater than the short-term range, such as greater than 1 month, greater than 3 months, the last 6 months, or longer, etc.). A low value can include a value in a lower percentage (e.g., at or below a mean or median, below the upper quartile, etc.). A medium value can, in certain examples, include a value between the upper and lower quartiles or within a threshold percentage of a mean or median, etc. In other examples, values can be determined with respect to clinical or population values, in certain examples, further respective to matching patient demographics (e.g., age, sex, comorbidities, etc.) or type of medical device (e.g., CRT-D device, ICD device, etc.), etc.

In an example, determinations described herein can be used to change device behavior, trigger additional sensing, data processing, storage, or transmission, or otherwise alter one or more modes, processes, or functions of medical devices associated with such determinations. For example, determinations can require data over a substantial time period (e.g., multiple days, weeks, a month or more, etc.). Such determinations can be initially determined by the device at yearly or semi-yearly (e.g., every 6 months, every 3 months, etc.) by default, or triggered by worsening patient status or upon instruction from a clinician or caregiver, etc. In a first example, an assessment circuit can determine one or more indications quarterly, consuming a default amount of device resources. If the quarterly determination exceeds one or more of a patient-specific or population threshold, the assessment circuit can alter device functionality to increase the frequency of making such determinations, increasing the use of device resources, in certain examples reducing device lifespan, but providing additional monitoring and determinations. In other examples, if a determination exceeds one or more thresholds, additional sensing can be triggered, such as enabling additional sensors, or sensing enabled sensors with a higher resolution or sampling frequency, storing more information, and communicating more information outside of the device, such as to an external programmer, or increasing the frequency of communication outside of the device, increasing the use of device resources, in certain examples reducing device lifespan, but providing additional monitoring and determinations.

In certain examples, determinations described herein can include one or more determined risk curves illustrating determined risks at different time periods into the future, such as a determined risk of mortality (e.g., cardiovascular death), a determined risk of heart failure hospitalization, etc. Information about the determined risks or the determined risk curves or portions of the determined risk curves themselves can be provided to a user, such as to a patient, clinician, caregiver, etc., or can be used to make one or more device changes, such as described herein (e.g., therapies, treatments, device settings, etc.), or trigger one or more other processes or notifications, etc.

### Composite Indications of Patient Condition

Indications of patient condition can include single-feature determinations based on a single feature or measure of a single type of physiologic information, or separately a composite determination based on a combination of physiologic information, such as two or more separate features of physiologic measures. In addition, indications of patient condition can be device-based, such as determined using physiologic information detected from the patient using the one or more ambulatory medical devices without input of clinical information about the patient separate from that detected or sensed physiologic information. In other examples, indications of patient condition can be a combination of device-based and clinical-based information of the patient, such as clinician diagnosis or determination of risk, patient history, patient age, comorbidities, prior hospitalization, type of implanted device, etc. In certain examples, separate determinations can be made for different combinations of clinical information.

One example of a composite indication is a HeartLogic™ index, a HeartLogic™ in-alert time, or one or more other composite measurements or measures thereof. The HeartLogic™ index is a composite indication of patient condition determined using different combinations or weightings of physiologic information, including two or more of S1 heart sounds, S3 heart sounds, thoracic impedance, activity information, respiration information, and nighttime heart rate (nHR). The HeartLogic™ index can be indicative of a heart failure status, a risk a heart failure event (e.g., within in a given time period), or a worsening of the heart failure status or risk of heart failure event in the patient over time. The HeartLogic™ in-alert time is a measure of time that the HeartLogic™ index is above an alert threshold.

In certain examples, the different combinations or weightings of physiologic information used to determine the HeartLogic™ index can be adjusted or determined based on a risk stratifier. In certain examples, the risk stratifier can be determined as a different combination of physiologic information, including one or more of S3, respiratory rate, and time active (e.g., an amount of time at a specific activity level above a mean activity level of the patient or a specific threshold, etc.). For example, if the risk stratifier is low, or below a first threshold, the HeartLogic™ index can be determined using a first combination of physiologic information. If the risk stratifier is high, or above a second threshold, the HeartLogic™ index can be determined using a second combination of physiologic information, such as additional information than included in the first combination (e.g., the first combination and the second combination, etc.). If the risk stratifier is between the first and second thresholds, the HeartLogic™ index can be determined using the first combination and one or more metrics or components of the second combination, or using the first combination and the second combination, but with the second combination having less weight than if the risk stratifier is above the second threshold (e.g., using less of the second combination than the first combination).

In an example, the HeartLogic™ index and in-alert time can include worsening heart failure or physiologic event detection, including risk indication or stratification, such as that disclosed in the commonly assigned An et al. U.S. Pat. No. 9,968,266 entitled “RISK STRATIFICATION BASED HEART FAILURE DETECTION ALGORITHM,” or in the commonly assigned An et al. U.S. Pat. No. 9,622,664 entitled “METHODS AND APPARATUS FOR DETECTING HEART FAILURE DECOMPENSATION EVENT AND STRATIFYING THE RISK OF THE SAME,” or in the commonly assigned Thakur et al. U.S. Pat. No. 10,660,577 entitled “SYSTEMS AND METHODS FOR DETECTING WORSENING HEART FAILURE,” or in the commonly assigned An et al. U.S. Patent Application No. 2014/0031643 entitled “HEART FAILURE PATIENT STRATIFICATION,” or in the commonly assigned Thakur et al. U.S. Pat. No. 10,085,696 entitled “DETECTION OF WORSENING HEART FAILURE EVENTS USING HEART SOUNDS,” each of which are hereby incorporated by reference in their entireties, including their disclosures of heart failure and worsening heart failure detection, heart failure risk indication detection, and stratification of the same, etc.

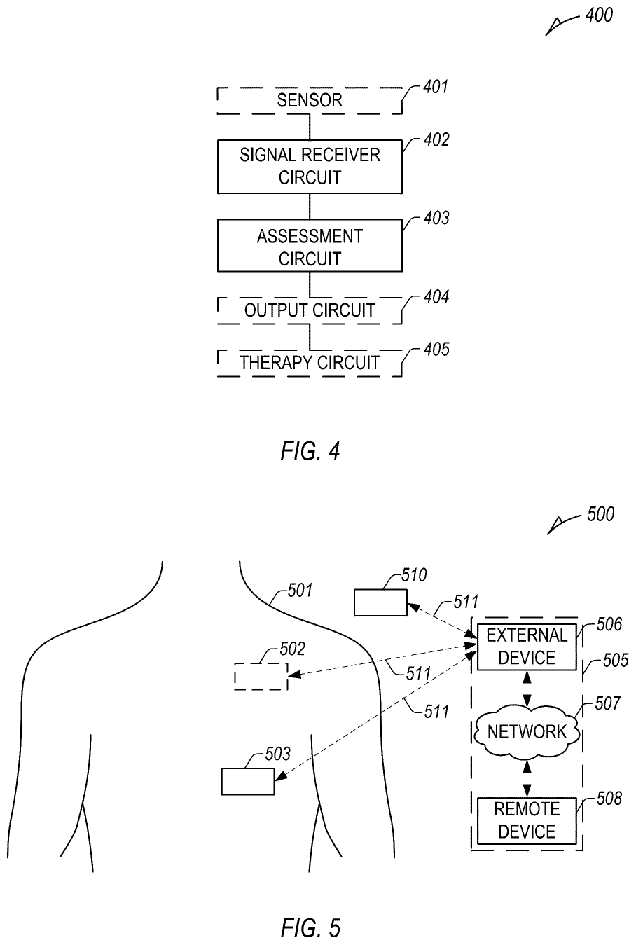

FIG. 4 illustrates an example system 400 (e.g., a medical device system). In an example, one or more aspects of the system 400 can be a component of, or communicatively coupled to, a medical device, such as an implantable medical device (IMD), an insertable cardiac monitor (ICM), an ambulatory medical device (AMD), etc. The system 400 can be configured to monitor, detect, or treat various physiologic conditions of the body, such as cardiac conditions associated with a reduced ability of a heart to sufficiently deliver blood to a body, including heart failure, arrhythmias, dyssynchrony, etc., or one or more other physiologic conditions and, in certain examples, can be configured to provide electrical stimulation or one or more other therapies or treatments to the patient.

The system 400 can include a single medical device or a plurality of medical devices implanted in a body of a patient or otherwise positioned on or about the patient to monitor patient physiologic information of the patient using information from one or more sensors, such as a sensor 401. In an example, the sensor 401 can include one or more of: a respiration sensor configured to receive respiration information (e.g., a respiratory rate, a respiration volume (tidal volume), etc.); an acceleration sensor (e.g., an accelerometer, a microphone, etc.) configured to receive cardiac acceleration information (e.g., cardiac vibration information, pressure waveform information, heart sound information, endocardial acceleration information, acceleration information, activity information, posture information, etc.); an impedance sensor (e.g., an intrathoracic impedance sensor, a transthoracic impedance sensor, a thoracic impedance sensor, etc.) configured to receive impedance information, a cardiac sensor configured to receive cardiac electrical information; an activity sensor configured to receive information about a physical motion (e.g., activity, steps, etc.); a posture sensor configured to receive posture or position information; a pressure sensor configured to receive pressure information; a plethysmograph sensor (e.g., a photoplethysmography sensor, etc.); a chemical sensor (e.g., an electrolyte sensor, a pH sensor, an anion gap sensor, a potassium sensor, a creatinine sensor, etc.); a temperature sensor; a skin elasticity sensor, or one or more other sensors configured to receive physiologic information of the patient.

The example system 400 can include a signal receiver circuit 402 and an assessment circuit 403. The signal receiver circuit 402 can be configured to receive physiologic information of a patient (or group of patients) from the sensor 401. The assessment circuit 403 can be configured to receive information from the signal receiver circuit 402, and to determine one or more parameters (e.g., physiologic parameters, stratifiers, etc.) or existing or changed patient conditions (e.g., indications of patient dehydration, respiratory condition, cardiac condition (e.g., heart failure, arrhythmia), sleep disordered breathing, etc.) using the received physiologic information, such as described herein. The physiologic information can include, among other things, cardiac electrical information, impedance information, respiration information, heart sound information, activity information, posture information, temperature information, or one or more other types of physiologic information. In an example, the signal receiver circuit 402 can include the sensor 401. In other examples, the signal receiver circuit can be coupled to or a component of the assessment circuit 403.

In certain examples, the assessment circuit 403 can aggregate information from multiple sensors or devices, detect various events using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

In certain examples, such as to detect an improved or worsening patient condition, some initial assessment is often required to establish a baseline level or condition from one or more sensors or physiologic information. Subsequent detection of a deviation from the baseline level or condition can be used to determine the improved or worsening patient condition. However, in other examples, the amount of variation or change (e.g., relative or absolute change) in physiologic information over different time periods can used to determine a risk of an adverse medical event, or to predict or stratify the risk of the patient experiencing an adverse medical event (e.g., a heart failure event) in a period following the detected change, in combination with or separate from any baseline level or condition.

Changes in different physiologic information can be aggregated and weighted based on one or more patient-specific stratifiers and, in certain examples, compared to one or more thresholds, for example, having a clinical sensitivity and specificity across a target population with respect to a specific condition (e.g., heart failure), etc., and one or more specific time periods, such as daily values, short term averages (e.g., daily values aggregated over a number of days), long term averages (e.g., daily values aggregated over a number of short term periods or a greater number of days (sometimes different (e.g., non-overlapping) days than used for the short term average)), etc.

In certain examples, the assessment circuit 403 can aggregate information from multiple sensors or devices, detect various events using information from each sensor or device separately or in combination, update a detection status for one or more patients based on the information, and transmit a message or an alert to one or more remote devices that a detection for the one or more patients has been made or that information has been stored or transmitted, such that one or more additional processes or systems can use the stored or transmitted detection or information for one or more other review or processes.

In certain examples, such as to detect an improved or worsening patient condition, some initial assessment is often required to establish a baseline level or condition from one or more sensors or physiologic information. Subsequent detection of a deviation from the baseline level or condition can be used to determine the improved or worsening patient condition. However, in other examples, the amount of variation or change (e.g., relative or absolute change) in physiologic information over different time periods can used to determine a risk of an adverse medical event, or to predict or stratify the risk of the patient experiencing an adverse medical event (e.g., a heart failure event) in a period following the detected change, in combination with or separate from any baseline level or condition.

Changes in different physiologic information can be aggregated and weighted based on one or more patient-specific stratifiers and, in certain examples, compared to one or more thresholds, for example, having a clinical sensitivity and specificity across a target population with respect to a specific condition (e.g., heart failure), etc., and one or more specific time periods, such as daily values, short term averages (e.g., daily values aggregated over a number of days), long term averages (e.g., daily values aggregated over a number of short term periods or a greater number of days (sometimes different (e.g., non-overlapping) days than used for the short term average)), etc.

The system 400 can include an output circuit 404 configured to provide an output to a user, or to cause an output to be provided to a user, such as through an output, a display, or one or more other user interface, the output including a score, a trend, an alert, or other indication. In other examples, the output circuit 404 can be configured to provide an output to another circuit, machine, or process, such as a therapy circuit 405 (e.g., a cardiac resynchronization therapy (CRT) circuit, a chemical therapy circuit, a stimulation circuit, etc.), etc., to control, adjust, or cease a therapy of a medical device, a drug delivery system, etc., or otherwise alter one or more processes or functions of one or more other aspects or components of a medical device system, such as one or more CRT parameters, drug delivery, dosage determinations or recommendations, etc. In an example, the therapy circuit 405 can include one or more of a stimulation control circuit, a cardiac stimulation circuit, a neural stimulation circuit, a dosage determination or control circuit, etc. In other examples, the therapy circuit 405 can be controlled by the assessment circuit 403, or one or more other circuits, etc. In certain examples, the assessment circuit 403 can include the output circuit 404 or can be configured to determine the output to be provided by the output circuit 404, while the output circuit 404 can provide the signals that cause the user interface to provide the output to the user based on the output determined by the assessment circuit 403.

FIG. 5 illustrates an example patient management system 500 and portions of an environment in which the patient management system 500 may operate. The patient management system 500 can perform a range of activities, including remote patient monitoring and diagnosis of a disease condition. Such activities can be performed proximal to a patient 501, such as in a patient home or office, through a centralized server, such as in a hospital, clinic, or physician office, or through a remote workstation, such as a secure wireless mobile computing device.

The patient management system 500 can include one or more medical devices, an external system 505, and a communication link 511 providing for communication between the one or more ambulatory medical devices and the external system 505. The one or more medical devices can include an ambulatory medical device (AMD), such as an implantable medical device (IMD) 502, a wearable medical device 503, or one or more other implantable, leadless, subcutaneous, external, wearable, or medical devices configured to monitor, sense, or detect information from, determine physiologic information about, or provide one or more therapies to treat various conditions of the patient 501, such as one or more cardiac or non-cardiac conditions (e.g., dehydration, sleep disordered breathing, etc.).

In an example, the implantable medical device 502 can include one or more cardiac rhythm management devices implanted in a chest of a patient, having a lead system including one or more transveous, subcutaneous, or non-invasive leads or catheters to position one or more electrodes or other sensors (e.g., a heart sound sensor) in, on, or about a heart or one or more other position in a thorax, abdomen, or neck of the patient 501. In another example, the implantable medical device 502 can include a monitor implanted, for example, subcutaneously in the chest of patient 501, the implantable medical device 502 including a housing containing circuitry and, in certain examples, one or more sensors, such as a temperature sensor, etc.

Cardiac rhythm management devices, such as insertable cardiac monitors, pacemakers, defibrillators, or cardiac resynchronizers, include implantable or subcutaneous devices having hermetically sealed housings configured to be implanted in a chest of a patient. The cardiac rhythm management device can include one or more leads to position one or more electrodes or other sensors at various locations in or near the heart, such as in one or more of the atria or ventricles of a heart, etc. Accordingly, cardiac rhythm management devices can include aspects located subcutaneously, though proximate the distal skin of the patient, as well as aspects, such as leads or electrodes, located near one or more organs of the patient. Separate from, or in addition to, the one or more electrodes or other sensors of the leads, the cardiac rhythm management device can include one or more electrodes or other sensors (e.g., a pressure sensor, an accelerometer, a gyroscope, a microphone, etc.) powered by a power source in the cardiac rhythm management device. The one or more electrodes or other sensors of the leads, the cardiac rhythm management device, or a combination thereof, can be configured detect physiologic information from the patient, or provide one or more therapies or stimulation to the patient.

Implantable devices can additionally or separately include leadless cardiac pacemakers (LCPs), small (e.g., smaller than traditional implantable cardiac rhythm management devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart without traditional lead or implantable cardiac rhythm management device complications (e.g., required incision and pocket, complications associated with lead placement, breakage, or migration, etc.). In certain examples, leadless cardiac pacemakers can have more limited power and processing capabilities than a traditional cardiac rhythm management device; however, multiple leadless cardiac pacemakers can be implanted in or about the heart to detect physiologic information from, or provide one or more therapies or stimulation to, one or more chambers of the heart. The multiple leadless cardiac pacemaker can communicate between themselves, or one or more other implanted or external devices.

The implantable medical device 502 can include a signal receiver circuit or an assessment circuit configured to detect or determine specific physiologic information of the patient 501, or to determine one or more conditions or provide information or an alert to a user, such as the patient 501 (e.g., a patient), a clinician, or one or more other caregivers or processes, such as described herein. The implantable medical device 502 can alternatively or additionally be configured as a therapeutic device configured to treat one or more medical conditions of the patient 501. The therapy can be delivered to the patient 501 via the lead system and associated electrodes or using one or more other delivery mechanisms. The therapy can include delivery of one or more drugs to the patient 501, such as using the implantable medical device 502 or one or more of the other ambulatory medical devices, etc. In some examples, therapy can include CRT for rectifying dyssynchrony and improving cardiac function in heart failure patients. In other examples, the implantable medical device 502 can include a drug delivery system, such as a drug infusion pump to deliver drugs to the patient for managing arrhythmias or complications from arrhythmias, hypertension, hypotension, or one or more other physiologic conditions. In other examples, the implantable medical device 502 can include one or more electrodes configured to stimulate the nervous system of the patient or to provide stimulation to the muscles of the patient airway, etc.

The wearable medical device 503 can include one or more wearable or external medical sensors or devices (e.g., automatic external defibrillators (AEDs), Holter monitors, patch-based devices, smart watches, smart accessories, wrist- or finger-worn medical devices, such as a finger-based photoplethysmography sensor, etc.).

The external system 505 can include a dedicated hardware/software system, such as a programmer, a remote server-based patient management system, or alternatively a system defined predominantly by software running on a standard personal computer. The external system 505 can manage the patient 501 through the implantable medical device 502 or one or more other ambulatory medical devices connected to the external system 505 via a communication link 511. In other examples, the implantable medical device 502 can be connected to the wearable medical device 503, or the wearable medical device 503 can be connected to the external system 505, via the communication link 511. This can include, for example, programming the implantable medical device 502 to perform one or more of acquiring physiologic data, performing at least one self-diagnostic test (such as for a device operational status), analyzing the physiologic data, or optionally delivering or adjusting a therapy for the patient 501. Additionally, the external system 505 can send information to, or receive information from, the implantable medical device 502 or the wearable medical device 503 via the communication link 511. Examples of the information can include real-time or stored physiologic data from the patient 501, diagnostic data, such as detection of patient hydration status, hospitalizations, responses to therapies delivered to the patient 501, or device operational status of the implantable medical device 502 or the wearable medical device 503 (e.g., battery status, lead impedance, etc.). The communication link 511 can be an inductive telemetry link, a capacitive telemetry link, or a radio frequency (RF) telemetry link, or wireless telemetry based on, for example, “strong” Bluetooth or IEEE 802.11 wireless fidelity “Wi-Fi” interfacing standards. Other configurations and combinations of patient data source interfacing are possible.

The external system 505 can include an external device 506 in proximity of the one or more ambulatory medical devices, and a remote device 508 in a location relatively distant from the one or more ambulatory medical devices, in communication with the external device 506 via a communication network 507. Examples of the external device 506 can include a medical device programmer. The remote device 508 can be configured to evaluate collected patient or patient information and provide alert notifications, among other possible functions. In an example, the remote device 508 can include a centralized server acting as a central hub for collected data storage and analysis from a number of different sources. Combinations of information from the multiple sources can be used to make determinations and update individual patient status or to adjust one or more alerts or determinations for one or more other patients. The server can be configured as a uni-, multi-, or distributed computing and processing system. The remote device 508 can receive data from multiple patients. The data can be collected by the one or more ambulatory medical devices, among other data acquisition sensors or devices associated with the patient 501. The server can include a memory device to store the data in a patient database. The server can include an alert analyzer circuit to evaluate the collected data to determine if specific alert condition is satisfied. Satisfaction of the alert condition may trigger a generation of alert notifications, such to be provided by one or more human-perceptible user interfaces. In some examples, the alert conditions may alternatively or additionally be evaluated by the one or more ambulatory medical devices, such as the implantable medical device. By way of example, alert notifications can include a Web page update, phone or pager call, E-mail, SMS, text, or “Instant” message, as well as a message to the patient and a simultaneous direct notification to emergency services and to the clinician. Other alert notifications are possible. The server can include an alert prioritizer circuit configured to prioritize the alert notifications. For example, an alert of a detected medical event can be prioritized using a similarity metric between the physiologic data associated with the detected medical event to physiologic data associated with the historical alerts.

The remote device 508 may additionally include one or more locally configured clients or remote clients securely connected over the communication network 507 to the server. Examples of the clients can include personal desktops, notebook computers, mobile devices, or other computing devices. System users, such as clinicians or other qualified medical specialists, may use the clients to securely access stored patient data assembled in the database in the server, and to select and prioritize patients and alerts for health care provisioning. In addition to generating alert notifications, the remote device 508, including the server and the interconnected clients, may also execute a follow-up scheme by sending follow-up requests to the one or more ambulatory medical devices, or by sending a message or other communication to the patient 501 (e.g., the patient), clinician or authorized third party as a compliance notification.

The communication network 507 can provide wired or wireless interconnectivity. In an example, the communication network 507 can be based on the Transmission Control Protocol/Internet Protocol (TCP/IP) network communication specification, although other types or combinations of networking implementations are possible. Similarly, other network topologies and arrangements are possible.

One or more of the external device 506 or the remote device 508 can output the detected medical events to a system user, such as the patient or a clinician, or to a process including, for example, an instance of a computer program executable in a microprocessor. In an example, the process can include an automated generation of recommendations for therapy, or a recommendation for further diagnostic test or treatment. In an example, the external device 506 or the remote device 508 can include a respective display unit for displaying the physiologic or functional signals, or alerts, alarms, emergency calls, or other forms of warnings to signal the detection of arrhythmias. In some examples, the external system 505 can include a signal receiver circuit and an assessment circuit, such as an external data processor configured to analyze the physiologic or functional signals received by the one or more ambulatory medical devices, and to confirm or reject one or more determinations made by one or more ambulatory medical devices, such as the implantable medical device 502, the wearable medical device 503, etc., or make additional determinations, etc. Computationally intensive algorithms, such as machine-learning algorithms, can be implemented in the external data processor to process the data retrospectively to detect cardia arrhythmias.

Portions of the one or more ambulatory medical devices or the external system 505 can be implemented using hardware, software, firmware, or combinations thereof. Portions of the one or more ambulatory medical devices or the external system 505 can be implemented using an application-specific circuit that can be constructed or configured to perform one or more functions or can be implemented using a general-purpose circuit that can be programmed or otherwise configured to perform one or more functions. Such a general-purpose circuit can include a microprocessor or a portion thereof, a microcontroller or a portion thereof, or a programmable logic circuit, a memory circuit, a network interface, and various components for interconnecting these components. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. “Sensors” can include electronic circuits configured to receive information and provide an electronic output representative of such received information.

A therapy device 510 can be configured to send information to or receive information from one or more of the ambulatory medical devices or the external system 505 using the communication link 511. In an example, the one or more ambulatory medical devices, the external device 506, or the remote device 508 can be configured to control one or more parameters of the therapy device 510. The external system 505 can allow for programming the one or more ambulatory medical devices and can receive information about one or more signals acquired by the one or more ambulatory medical devices, such as can be received via a communication link 511. The external system 505 can include a local external implantable medical device programmer. The external system 505 can include a remote patient management system that can monitor patient status or adjust one or more therapies such as from a remote location.

In certain examples, event storage can be triggered, such as received physiologic information or in response to one or more detected events or determined parameters meeting or exceeding a threshold (e.g., a static threshold, a dynamic threshold, or one or more other thresholds based on patient or population information, etc.). Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected events can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, amount of memory, etc.). Storing multiple windows using this early detection leading up to a single event can provide full event assessment with power and cost savings, in contrast to the longer loop recorder windows. In addition, the early detection can trigger additional parameter computation or storage, at different resolution or sampling frequency, without unduly taxing finite system resources.

In certain examples, one or more alerts can be provided, such as to the patient, to a clinician, or to one or more other caregivers (e.g., using a patient smart watch, a cellular or smart phone, a computer, etc.), in certain examples, in response to the transition to the high-power mode, in response to the detected event or condition, or after updating or transmitting information from a first device to a remote device. In other examples, the medical device itself can provide an audible or tactile alert to warn the patient of the detected condition. For example, the patient can be alerted in response to a detected condition so they can engage in corrective action, such as sitting down, etc.

In certain examples, a therapy can be provided in response to the detected condition. For example, a pacing therapy can be provided, enabled, or adjusted, such as to disrupt or reduce the impact of the detected event. In other examples, delivery of one or more drugs (e.g., a vasoconstrictor, pressor drugs, etc.) can be triggered, provided, or adjusted, such as using a drug pump, in response to the detected condition, alone or in combination with a pacing therapy, such as that described above, for example, to increase arterial pressure, to maintain cardiac output, to disrupt or reduce the impact of the detected event, or combinations thereof.

In certain examples, physiologic information of a patient can be sensed using one or more sensors located within, on, or proximate to the patient, such as a cardiac sensor, a heart sound sensor, or one or more other sensors described herein. For example, cardiac electrical information of the patient can be sensed using a cardiac sensor. In other examples, cardiac acceleration information of the patient can be sensed using a heart sound sensor. The cardiac sensor and the heart sound sensor can be components of one or more (e.g., the same or different) medical devices (e.g., an implantable medical device, an ambulatory medical device, etc.). Timing metrics between different features (e.g., first and second cardiac features, etc.) can be determined, such as by a processing circuit of the cardiac sensor or one or more other medical devices or medical device components, etc. In certain examples, the timing metric can include an interval or metric between first and second cardiac features of a first cardiac interval of the patient (e.g., a duration of a cardiac cycle or interval, a QRS width, etc.) or between first and second cardiac features of respective successive first and second cardiac intervals of the patient. In an example, the first and second cardiac features include equivalent detected features in successive first and second cardiac intervals, such as successive R waves (e.g., an R-R interval, etc.) or one or more other features of the cardiac electrical signal, etc.

In an example, heart sound signal portions, or values of respective heart sound signals for a cardiac interval, can be detected as amplitudes occurring with respect to one or more cardiac electrical features or one or more energy values with respect to a window of the heart sound signal, often determined with respect to one or more cardiac electrical features. For example, the value and timing of an S1 signal can be detected using an amplitude or energy of the heart sound signal occurring at or about the R wave of the cardiac interval. An S4 signal portion can be determined, such as by a processing circuit of the heart sound sensor or one or more other medical devices or medical device components, etc. In certain examples, the S4 signal portion can include a filtered signal from an S4 window of a cardiac interval. In an example, the S4 interval can be determined as a set time period in the cardiac interval with respect to one or more other cardiac electrical or mechanical features, such as forward from one or more of the R wave, the T wave, or one or more features of a heart sound waveform, such as the first, second, or third heart sounds (S1, S2, S3), or backwards from a subsequent R wave or a detected S1 of a subsequent cardiac interval. In certain examples, the length of the S4 window can depend on heart rate or one or more other factors. In an example, the timing metric of the cardiac electrical information can be a timing metric of a first cardiac interval, and the S4 signal portion can be an S4 signal portion of the same first cardiac interval.

In an example, a heart sound parameter can include information of or about multiple of the same heart sound parameter or different combinations of heart sound parameters over one or more cardiac cycles or a specified time period (e.g., 1 minute, 1 hour, 1 day, 1 week, etc.). For example, a heart sound parameter can include a composite S1 parameter representative of a plurality of S1 parameters, for example, over a certain time period (e.g., a number of cardiac cycles, a representative time period, etc.).

In an example, the heart sound parameter can include an ensemble average of a particular heart sound over a heart sound waveform, such as that disclosed in the commonly assigned Siejko et al. U.S. Pat. No. 7,115,096 entitled “THIRD HEART SOUND ACTIVITY INDEX FOR HEART FAILURE MONITORING,” or in the commonly assigned Patangay et al. U.S. Pat. No. 7,853,327 entitled “HEART SOUND TRACKING SYSTEM AND METHOD,” each of which are hereby incorporated by reference in their entireties, including their disclosures of ensemble averaging an acoustic signal and determining a particular heart sound of a heart sound waveform. In other examples, the signal receiver circuit can receive the at least one heart sound parameter or composite parameter, such as from a heart sound sensor or a heart sound sensor circuit.

In an example, cardiac electrical information of the patient can be received, such as using a signal receiver circuit of a medical device, from a cardiac sensor (e.g., one or more electrodes, etc.) or cardiac sensor circuit (e.g., including one or more amplifier or filter circuits, etc.). In an example, the received cardiac electrical information can include the timing metric between the first and second cardiac features of the patient.

In an example, cardiac acceleration information of the patient can be received, such as using the same or different signal receiver circuit of the medical device, from a heart sound sensor (e.g., an accelerometer, etc.) or heart sound sensor circuit (e.g., including one or more amplifier or filter circuits, etc.). In an example, the received cardiac acceleration information can include the S4 signal portion occurring between the first and second cardiac features of the patient. In certain examples, additional physiologic information can be received, such as one or more of heart rate information, activity information of the patient, or posture information of the patient, from one or more other sensor or sensor circuits.

In certain examples, a high-power mode can be in contrast to a low-power mode, and can include one or more of: enabling one or more additional sensors, transitioning from a low-power sensor or set of sensors to a higher-power sensor or set of sensors, triggering additional sensing from one or more additional sensors or medical devices, increasing a sensing frequency or a sensing or storage resolution, increasing an amount of data to be collected, communicated (e.g., from a first medical device to a second medical device, etc.), or stored, triggering storage of currently available information from a loop recorder in long-term storage or increasing the storage capacity or time period of a loop recorder, or otherwise altering device behavior to capture additional or higher-resolution physiologic information or perform more processing, etc.

Additionally, or alternatively, event storage can be triggered. Information sensed or recorded in the high-power mode can be transitioned from short-term storage, such as in a loop recorder, to long-term or non-volatile memory, or in certain examples, prepared for communication to an external device separate from the medical device. In an example, cardiac electrical or cardiac mechanical information leading up to and in certain examples including the detected event (e.g., a heart failure event, an arrhythmia event, etc.) can be stored, such as to increase the specificity of detection. In an example, multiple loop recorder windows (e.g., 2-minute windows) can be stored sequentially. In systems without early detection, to record this information, a loop recorder with a longer time period would be required at substantial additional cost (e.g., power, processing resources, component cost, etc.).

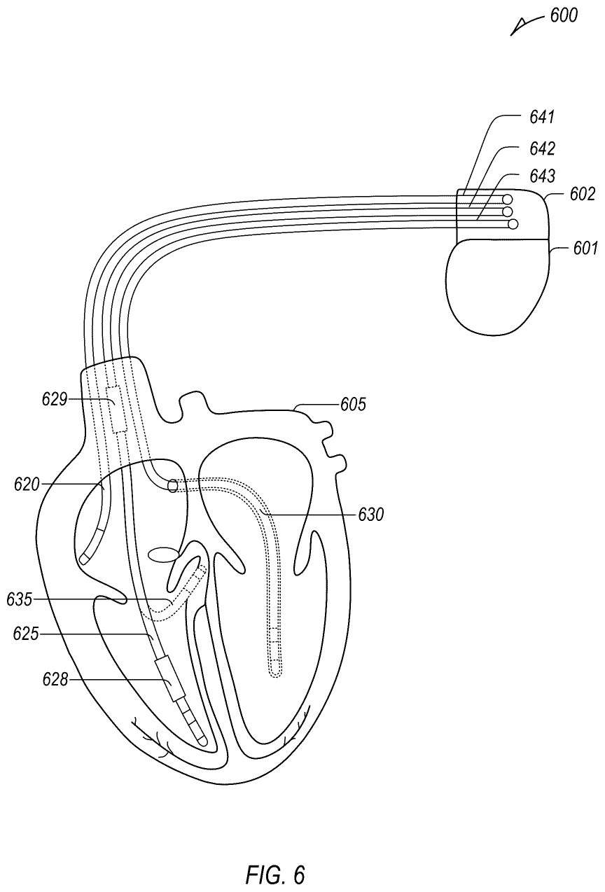

FIG. 6 illustrates an example implantable medical device (IMD) 600 electrically coupled to a heart 605, such as through one or more leads coupled to the implantable medical device 600 through one or more lead ports, including first, second, or third lead ports 641, 642, 643 in a header 602 of the implantable medical device 600. In an example, the implantable medical device 600 can include an antenna, such as in the header 602, configured to enable communication with an external system and one or more electronic circuits (e.g., an assessment circuit, etc.) in a hermetically sealed housing (CAN) 601.

The implantable medical device 600 may include an implantable cardiac monitor (ICM), pacemaker, defibrillator, cardiac resynchronizer, or other subcutaneous implantable medical device or cardiac rhythm management (CRM) device configured to be implanted in a chest of a subject, having one or more leads to position one or more electrodes or other sensors at various locations in or near the heart 605, such as in one or more of the atria or ventricles. Separate from, or in addition to, the one or more electrodes or other sensors of the leads, the implantable medical device 600 can include one or more electrodes or other sensors (e.g., a pressure sensor, an accelerometer, a gyroscope, a microphone, etc.) powered by a power source in the implantable medical device 600. The one or more electrodes or other sensors of the leads, the implantable medical device 600, or a combination thereof, can be configured detect physiologic information from, or provide one or more therapies or stimulation to, the patient.

Implantable devices can additionally include a leadless cardiac pacemaker (LCP), small (e.g., smaller than traditional implantable devices, in certain examples having a volume of about 1 cc, etc.), self-contained devices including one or more sensors, circuits, or electrodes configured to monitor physiologic information (e.g., heart rate, etc.) from, detect physiologic conditions (e.g., tachycardia) associated with, or provide one or more therapies or stimulation to the heart 605 without traditional lead or implantable device complications (e.g., required incision and pocket, complications associated with lead placement, breakage, or migration, etc.). In certain examples, a leadless cardiac pacemaker can have more limited power and processing capabilities than a traditional CRM device; however, multiple leadless cardiac pacemaker devices can be implanted in or about the heart to detect physiologic information from, or provide one or more therapies or stimulation to, one or more chambers of the heart. The multiple LCP devices can communicate between themselves, or one or more other implanted or external devices.

The implantable medical device 600 can include one or more electronic circuits configured to sense one or more physiologic signals, such as an electrogram or a signal representing mechanical function of the heart 605. In certain examples, the CAN 601 may function as an electrode such as for sensing or pulse delivery. For example, an electrode from one or more of the leads may be used together with the CAN 601 such as for unipolar sensing of an electrogram or for delivering one or more pacing pulses. A defibrillation electrode (e.g., the first defibrillation coil electrode 628, the second defibrillation coil electrode 629, etc.) may be used together with the CAN 601 to deliver one or more cardioversion/defibrillation pulses.

In an example, the implantable medical device 600 can sense impedance such as between electrodes located on one or more of the leads or the CAN 601. The implantable medical device 600 can be configured to inject current between a pair of electrodes, sense the resultant voltage between the same or different pair of electrodes, and determine impedance, such as using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing, etc. In an example, the implantable medical device 600 can be configured to inject current between an electrode on one or more of the first, second, third, or fourth leads 620, 625, 630, 635 and the CAN 601, and to sense the resultant voltage between the same or different electrodes and the CAN 601.

The implantable medical device 600 can integrate one or more other physiologic sensors to sense one or more other physiologic signals, such as one or more of heart rate, heart rate variability, intrathoracic impedance, intracardiac impedance, arterial pressure, pulmonary artery pressure, RV pressure, LV coronary pressure, coronary blood temperature, blood oxygen saturation, one or more heart sounds, physical activity or exertion level, physiologic response to activity, posture, respiration, body weight, or body temperature. The arrangement and functions of these leads and electrodes are described above by way of example and not by way of limitation. Depending on the need of the patient and the capability of the implantable device, other arrangements and uses of these leads and electrodes are.

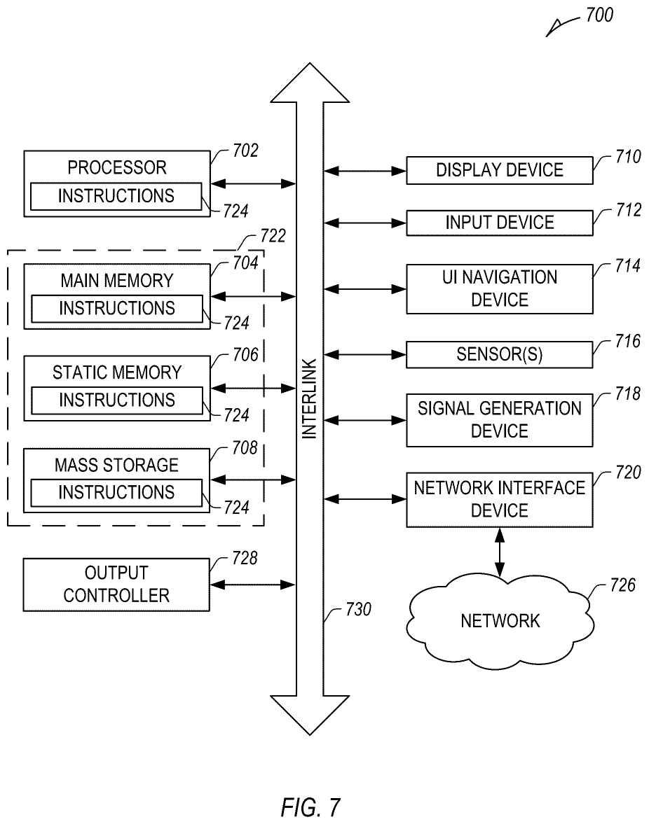

FIG. 7 illustrates a block diagram of an example machine 700 upon which any one or more of the techniques (e.g., methodologies) discussed herein may perform. Portions of this description may apply to the computing framework of one or more of the medical devices described herein, such as the implantable medical device, the external programmer, etc. Further, as described herein with respect to medical device components, systems, or machines, such may require regulatory-compliance not capable by generic computers, components, or machinery.

Examples, as described herein, may include, or may operate by, logic or a number of components, or mechanisms in the machine 700. Circuitry (e.g., processing circuitry, an assessment circuit, etc.) is a collection of circuits implemented in tangible entities of the machine 700 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine-readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine-readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 700 follow.

In alternative embodiments, the machine 700 may operate as a standalone device or may be connected (e.g., networked) to other machines. In a networked deployment, the machine 700 may operate in the capacity of a server machine, a client machine, or both in server-client network environments. In an example, the machine 700 may act as a peer machine in peer-to-peer (P2P) (or other distributed) network environment. The machine 700 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine 700 (e.g., computer system) may include a hardware processor 702 (e.g., a central processing unit (CPU), a graphics processing unit (GPU), a hardware processor core, or any combination thereof), a main memory 704, a static memory 706 (e.g., memory or storage for firmware, microcode, a basic-input-output (BIOS), unified extensible firmware interface (UEFI), etc.), and mass storage 708 (e.g., hard drive, tape drive, flash storage, or other block devices) some or all of which may communicate with each other via an interlink 730 (e.g., bus). The machine 700 may further include a display unit 710, an alphanumeric input device 712 (e.g., a keyboard), and a user interface (UI) navigation device 714 (e.g., a mouse). In an example, the display unit 710, input device 712, and UI navigation device 714 may be a touch screen display. The machine 700 may additionally include a signal generation device 718 (e.g., a speaker), a network interface device 720, and one or more sensors 716, such as a global positioning system (GPS) sensor, compass, accelerometer, or one or more other sensors. The machine 700 may include an output controller 728, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 702, the main memory 704, the static memory 706, or the mass storage 708 may be, or include, a machine-readable medium 722 on which is stored one or more sets of data structures or instructions 724 (e.g., software) embodying or utilized by any one or more of the techniques or functions described herein. The instructions 724 may also reside, completely or at least partially, within any of registers of the processor 702, the main memory 704, the static memory 706, or the mass storage 708 during execution thereof by the machine 700. In an example, one or any combination of the hardware processor 702, the main memory 704, the static memory 706, or the mass storage 708 may constitute the machine-readable medium 722. While the machine-readable medium 722 is illustrated as a single medium, the term “machine-readable medium” may include a single medium or multiple media (e.g., a centralized or distributed database, and/or associated caches and servers) configured to store the one or more instructions 724.

The term “machine-readable medium” may include any medium that is capable of storing, encoding, or carrying instructions for execution by the machine 700 and that cause the machine 700 to perform any one or more of the techniques of the present disclosure, or that is capable of storing, encoding, or carrying data structures used by or associated with such instructions. Non-limiting machine-readable medium examples may include solid-state memories, optical media, magnetic media, and signals (e.g., radio frequency signals, other photon-based signals, sound signals, etc.). In an example, a non-transitory machine-readable medium comprises a machine-readable medium with a plurality of particles having invariant (e.g., rest) mass, and thus are compositions of matter. Accordingly, non-transitory machine-readable media are machine-readable media that do not include transitory propagating signals. Specific examples of non-transitory machine-readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 724 may be further transmitted or received over a communications network 726 using a transmission medium via the network interface device 720 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an example, the network interface device 720 may include one or more physical jacks (e.g., Ethernet, coaxial, or phone jacks) or one or more antennas to connect to the communications network 726. In an example, the network interface device 720 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding, or carrying instructions for execution by the machine 700, and includes digital or analog communications signals or other intangible medium to facilitate communication of such software. A transmission medium is a machine-readable medium.

Various embodiments are illustrated in the figures above. One or more features from one or more of these embodiments may be combined to form other embodiments. Method examples described herein can be machine or computer-implemented at least in part. Some examples may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times.

The above detailed description is intended to be illustrative, and not restrictive. The scope of the disclosure should, therefore, be determined with references to the appended claims, along with the full scope of equivalents to which such claims are entitled.

What is claimed is:

1. A medical device system comprising:
a signal receiver circuit configured to receive physiologic information of a patient sensed by an implantable medical device implanted in the patient; and
an assessment circuit configured to:
determine a representative value of the received physiologic information of the patient;
determine a hybrid or relative baseline for the patient using the received physiologic information;
determine an indication of patient condition as a weighted function of (1) the representative value of the received physiologic information, (2) an absolute baseline, and (3) the hybrid or relative determined relative baseline, with a weight of at least one the determined absolute baseline or the determined relative baseline changing with time relative to a time of implant of the implantable medical device; and
provide the determined indication of patient condition to a user or process.

2. The medical device system of claim 1,
wherein the absolute baseline includes an imputed baseline stored or received by the medical device system.

3. The medical device system of claim 2,
wherein to determine the representative value of the received physiologic information of the patient includes to determine a value representative of at least a portion of at least one day of the received physiologic information of the patient,
wherein to determine the relative baseline for the patient includes using information from the imputed baseline representative of a greater number of days than represented by the determined representative value.

4. The medical device system of claim 3,
wherein to determine the value representative of at least a portion of at least one day of the received physiologic information includes to determine a value representative of one to three days of the received physiologic information of the patient, including a most recent one to three days,
wherein the greater number of days includes at least 30 days prior to the one to three days of the received physiologic information of the patient, prior to or including the most recent one to three days,
wherein the assessment circuit is configured to provide an output of determined indication of patient condition to a user interface for display to the user or to a control circuit to control or adjust the process or function of the medical device system.

5. The medical device system of claim 3,
wherein the assessment circuit is configured to determine the absolute baseline using information about the patient, the implantable medical device, or the received physiologic information, wherein the absolute baseline is different than the determined relative baseline.

6. The medical device system of claim 5,
wherein the assessment circuit is configured to determine the absolute baseline using the received physiologic information of the patient over a time period smaller than the number of days used to determine the relative baseline.

7. The medical device system of claim 1,
wherein the weight of the absolute baseline decreases in time relative to the time of implant of the medical device.

8. The medical device system of claim 1,
wherein the weight of the relative baseline increases in time relative to the time of implant of the medical device.

9. The medical device system of claim 1,
wherein, in a first period after implant of the implantable medical device, the weight of the absolute baseline in the weighted function is greater than the weight of the determined relative baseline,
wherein, after a near-implant time period, the weight of the determined relative baseline in the weighted function is greater than the weight of the absolute baseline.

10. The medical device system of claim 9,
wherein to determine the indication of patient condition as the weighted function of (1) the representative value of the received physiologic information, (2) an absolute baseline, and (3) the determined relative baseline, the assessment circuit is configured to:
determine, in the first period, the indication of patient condition as a function of (1) the representative value of the received physiologic information and (2) the absolute baseline, without using (3) the determined relative baseline;
determine, after the near-implant time period, the indication of patient condition as a function of (1) the representative value of the received physiologic information and (3) the determined relative baseline, without using (2) the absolute baseline; and
determine, after the first period and before the near-implant time period, the indication of patient condition as the weighted function of (1) the representative value of the received physiologic information, (2) an absolute baseline, and (3) the determined relative baseline.

11. A method comprising:
receiving, using a signal receiver circuit, physiologic information of a patient sensed by an implantable medical device implanted in the patient;
determining, using an assessment circuit, a representative value of the received physiologic information of the patient;
determining, using the assessment circuit, a relative baseline for the patient using the received physiologic information;
determining, using the assessment circuit, an indication of patient condition as a weighted function of (1) the representative value of the received physiologic information, (2) an absolute baseline, and (3) the determined relative baseline, with a weight of at least one the determined absolute baseline or the determined relative baseline changing with time relative to a time of implant of the implantable medical device; and
providing, using the assessment circuit, the determined indication of patient condition to a user or process.

12. The method of claim 11,
wherein the absolute baseline includes an imputed baseline stored or received by the assessment circuit or the implantable medical device.

13. The method of claim 12,
wherein determining the representative value of the received physiologic information of the patient includes determining a value representative of at least a portion of at least one day of the received physiologic information of the patient,
wherein determining the relative baseline for the patient includes using information from the imputed baseline representative of a greater number of days than represented by the determined representative value.

14. The method of claim 13,
wherein determining the value representative of at least a portion of at least one day of the received physiologic information includes determining a value representative of one to three days of the received physiologic information of the patient, including a most recent one to three days,
wherein the greater number of days includes at least 30 days prior to the one to three days of the received physiologic information of the patient, prior to or including the most recent one to three days,
wherein providing the determined indication of patient condition includes providing an output of determined indication of patient condition to a user interface for display to the user or to a control circuit to control or adjust the process or function of a component of a medical device system.

15. The method of claim 13, comprising:
determining, using the assessment circuit, the absolute baseline using information about the patient, the implantable medical device, or the received physiologic information, wherein the absolute baseline is different than the determined relative baseline.

16. The method of claim 15,
wherein determining the absolute baseline includes using the received physiologic information of the patient over a time period smaller than the number of days used to determine the relative baseline.

17. The method of claim 11,
wherein the weight of the absolute baseline decreases in time relative to the time of implant of the medical device.

18. The method of claim 11,
wherein the weight of the relative baseline increases in time relative to the time of implant of the medical device.

19. The method of claim 11,
wherein, in a first period after implant of the implantable medical device, the weight of the absolute baseline in the weighted function is greater than the weight of the determined relative baseline,
wherein, after a near-implant time period, the weight of the determined relative baseline in the weighted function is greater than the weight of the absolute baseline.

20. The method of claim 19,
wherein determining the indication of patient condition as the weighted function of (1) the representative value of the received physiologic information, (2) an absolute baseline, and (3) the determined relative baseline, includes:
determining, in the first period, the indication of patient condition as a function of (1) the representative value of the received physiologic information and (2) the absolute baseline, without using (3) the determined relative baseline;
determining, after the near-implant time period, the indication of patient condition as a function of (1) the representative value of the received physiologic information and (3) the determined relative baseline, without using (2) the absolute baseline; and
determining, after the first period and before the near-implant time period, the indication of patient condition as the weighted function of (1) the representative value of the received physiologic information, (2) an absolute baseline, and (3) the determined relative baseline.
